(19) United States
(12) Patent Application Publication
(10) Pub. No.: US 2024/0254174 A1
(43) Pub. Date: Aug. 1, 2024

(54) RABIES VIRAL GLYCOPROTEIN COMPOSITIONS AND METHODS OF USE

(71) Applicant: University of Alaska Fairbanks, Fairbanks, AK (US)

(72) Inventor: MAEGAN WELTZIN, Fairbanks, AK (US)

(21) Appl. No.: 18/422,865

(22) Filed: Jan. 25, 2024

Related U.S. Application Data
(60) Provisional application No. 63/481,928, filed on Jan. 27, 2023.

(51) Int. Cl. C07K 14/46; A61K 47/64; A61P 25/24; C07K 14/005; C12N 15/113; A61K 38/00

(52) U.S. Cl. CPC C07K 14/46; A61K 47/6415; A61P 25/24; C07K 14/005; C12N 15/113; A61K 38/00; C07K 2319/55; C12N 2310/14; C12N 2310/3513; C12N 2320/32

(57) ABSTRACT

Disclosed are peptides comprising a first portion, a second portion and a third portion, wherein at least the second portion is a portion of a RVG peptide, and wherein at least one of the first or third portions is a nAChR subtype selective peptide In some aspects, the nAChR subtype selective peptide can be from an α-neurotoxin. Disclosed are peptides comprising a first portion, a second portion and a third portion, wherein the first portion is a nicotinic acetylcholine receptor (nAChR) subtype selective portion of an α-neurotoxin, wherein the second portion is a portion of a rabies viral glycoprotein (RVG) peptide, and wherein the third portion is a nicotinic acetylcholine receptor (nAChR) subtype selective portion of an α-neurotoxin. In some aspects, the RVG peptide is a cell penetrating portion. Disclosed are methods of using these peptides.

### CROSS-REFERENCE TO RELATED APPLICATIONS

This application claims the benefit of U.S. Provisional Patent Application No. 63/481,928, filed Jan. 27, 2023, which is incorporated by reference herein in its entirety.

### STATEMENT REGARDING FEDERALLY SPONSORED RESEARCH

This invention was made with government support under Grant Number P20GM103395 awarded by the National Institutes of Health. The government has certain rights in this invention.

### REFERENCE TO SEQUENCE LISTING

The Sequence Listing submitted Jan. 25, 2024 as a xml file named “21002.0108U2.xml,” created on Jan. 24, 2024, and having a size of 21,699 bytes is hereby incorporated by reference pursuant to 37 C.F.R. § 1.52(ex5).

### BACKGROUND

In light of the current opioid use epidemic, research in the area of alternative pain therapies is extremely important. Frequently found together, chronic neurological pain and depression are in part mediated by distinct nicotinic acetylcholine receptor (nAChR) subtypes located in the central and peripheral nervous systems (CNS, PNS). Thus, peptides that interact with specific subtypes of nAChRs located on the cell surface have great therapeutic potential. Small molecules that antagonize α9α10 and those that induce α7 desensitization have been shown to improve pain and depression-like symptoms. Conotoxin RgIA has proved to be extremely useful in the management of neuropathic and inflammatory pain via inhibition of 09010 nAChRs. However, bioavailability and stability limit the application of this molecule.

Cell penetrating peptides (CPPs) generally interact broadly with cellular macromolecules and lipid bilayers, not with a specific molecular target. Rabies viral glycoprotein (RVG) peptides have been used successfully as CPPs to transport therapeutics across the mouse blood brain barrier (BBB), but display low nAChR subtype selectivity.

What is needed are peptides with increased nAChR subtype selectivity and can penetrate into cells.

### BRIEF SUMMARY

Disclosed herein are peptides to increase nAChR subtype selectivity and cell penetration.

Disclosed are peptides comprising a first portion, a second portion and a third portion, wherein the first portion is a nicotinic acetylcholine receptor (nAChR) subtype selective portion of an α-neurotoxin, wherein the second portion is a portion of a rabies viral glycoprotein (RVG) peptide, and wherein the third portion is a nicotinic acetylcholine receptor (nAChR) subtype selective portion of an α-neurotoxin. In some aspects, the RVG peptide is a cell penetrating portion.

Disclosed are peptides comprising a first portion, a second portion and a third portion, wherein the first portion is an nAChR subtype selective portion of an α-neurotoxin, wherein the second portion is a portion of a RVG peptide, and wherein the third portion is a nAChR subtype selective portion of an α-neurotoxin, wherein the first portion comprises the amino acid sequence YRKMW (SEQ ID NO:7), YTKTW (SEQ ID NO:8), or YTIW (SEQ ID NO: 22), the second portion comprises the amino acid sequence MPENPRLGTS (SEQ ID NO:2) and the third portion comprises the sequence CDAFCSSRGKVVELG (SEQ ID NO:9), CDAFCSIRGKRVDLG (SEQ ID NO:10), or CDIFTNSRGKRASKG (SEQ ID NO:23).

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the first portion comprises an amino acid sequence with 40%, 45%, 50%, 55%, 60%, 65%, 70%, 75%, 80%, 85%, 90%, 95%, or 99% identity to YRKMW (SEQ ID NO:7) or YTKTW (SEQ ID NO:8). In some aspects, the second and third portions can be any of those disclosed herein.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the second portion comprises an amino acid sequence with 40%, 45%, 50%, 55%, 60%, 65%, 70%, 75%, 80%, 85%, 90%, 95%, or 99% identity to MPENPRLGTS (SEQ ID NO:2). In some aspects, the first and third portions can be any of those disclosed herein.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the third portion comprises an amino acid sequence with 40%, 45%, 50%, 55%, 60%, 65%, 70%, 75%, 80%, 85%, 90%, 95%, or 99% identity to CDAFCSSRGKVVELG (SEQ ID NO:9) or CDAFCSIRGKRVDLG (SEQ ID NO:10). In some aspects, the first and second portions can be any of those disclosed herein.

Disclosed here are peptides comprising the amino acid sequence YRKMWMPENPRLGTSCDAFCSSRGKVVELG (SEQ ID NO:11). Disclosed here are peptides comprising or consisting of an amino acid sequence having one or more amino acid substitutions at positions 3, 5, 14, 17, 19, 22, 23, or 26 of

(SEQ ID NO: 11)
YRKMWMPENPRLGTSCDAFCSSRGKVVELG.

Disclosed are nucleic acid sequences capable of encoding one or more of the disclosed peptides.

Also disclosed herein are pharmaceutical compositions comprising the disclosed peptides, nucleic acids vectors, or cells and a pharmaceutically acceptable carrier thereof.

Disclosed are methods of antagonizing an α7 and/or α9α10 nAChR response in a subject comprising administering any of the disclosed peptides to a subject in need thereof.

Disclosed are methods of inhibiting α7 and/or α9α10 nAChR in a subject comprising administering any of the disclosed peptides to a subject in need thereof.

Disclosed are methods of decreasing inflammation in a subject comprising administering any of the disclosed peptides to a subject in need thereof.

Disclosed are methods of treating or ameliorating a symptom of chronic pain in a subject comprising administering a composition comprising one or more of the disclosed peptides to a subject in need thereof.

Disclosed are methods of treating or ameliorating a symptom of depression in a subject comprising administering a composition comprising one or more of the disclosed peptides to a subject in need thereof.

Disclosed are methods of increasing nAChR subtype specificity comprising administering a composition comprising one of the disclosed peptides, wherein the peptide comprises or consists of the amino acid sequence having one or more amino acid substitutions at positions 3, 5, 14, 17, 19, 22, 23, or 26 of YRKMWMPENPRLGTSCDAFCSSRGKVVELG (SEQ ID NO:11), to a cell.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of the RVG peptide concentration response curves show that the peptide is not very selective for a specific nAChR subtype (N≥2, n≥5). α7 nAChR potency is 32±3 μM.

FIG. 2 shows a sequence comparison between the RVG peptide and α-neurotoxin loop II. Residues in Regions 1, 2, and 3 are identified, along with the used numbering scheme. Underlined residues have been previously shown to alter nAChR interactions, italicized residues are involved in selectivity, while bolded blue highlighted residues are proposed to be initially mutated in the lead fusion peptide.

FIGS. 3A and 3B show the lead RVG fusion peptide demonstrates high 07 and 09100 nAChR selectivity. FIG. 3A) Bar graph of our rapid screening outcomes that allows us to determine subtype selectivity (mean±SEM, N=1-2). FIG. 3B) Concentration response profiles of RVG and the lead peptide on 07 (left) and α9α10 (right) nAChRs demonstrating the lead peptide is more potent than the RVG peptide (RVG: α7 31±3 μM, α9α10 153±14 μM; lead peptide: α7 20±2 μM, α9α10 54±7 μM) (N=3).

FIGS. 4A-4D shows mutagenesis and molecular modeling of the RVG peptide. FIG. 4A and FIG. 4B) Several α7 nAChRs hot spot residues have been identified in the parent RVG peptide (mean±SEM; N=1-3). Modeled RVG (FIG. 4C) and lead fusion (FIG. 4D) peptides displaying amino acid hydrophobicity, and identified RVG hot spot residues.

FIGS. 5A-5D shows exemplary cytotoxicity outcomes for the parent and lead peptides on α7 nAChR transfected (FIG. 5A and FIG. 5B) and non-transfected (FIG. 5C and FIG. 5D) N2a cells. Cells were exposed to peptide for 24 hrs prior to performing the viability assay (mean±SEM, N=1-2).

FIG. 6 shows examples of changes in the predicted structure of the peptides disclosed herein and the functional measured effect.

FIGS. 7A-7B show accumulation of the lead CPP in α7-nAChR transfected N2a cells, which sharply contrasts the surface only labeling of α-btx-AF647. 7A. No phase image of α7-nAChR transfected cells with lead CPP-FITC found within the cells. 7A′. Image A with phase, highlighting lead CPP internalization. 7A”. 30° rotation of 7A′. 7B. No phase image showing α-btx-AF647 only on the surface of α7-nAChR transfected cells. 7B′. Image B but with phase. Note the lack of fluorescence inside the cells. 7B”. 30° rotation of B′. METHODS: Cells were exposed to drug (10 μM CPP, 800 nM α-btx based on IC₅₀ values) for 24 hr followed by live cell confocal microscopy (lead CPP-FITC: 495/519 nm (ex/em), α-btx-AF647: 653/668 run) (deconvoluted, ImageJ, N=3).

FIGS. 8A-8C show the lead CPP-FITC enters cells via α7-nAChRs. 8A&A′. The lead CPP-FITC readily interacts with N2a cells transfected with α7-nAChR. 8B&B′. Un-transfected cells bind α-btx-AF647, demonstrating N2a cells endogenously express α7-nAChRs. 8C&C′. Un-transfected HEK cells exposed to lead CPP-FITC demonstrating α7-nAChRs are required for cell targeting. Green hue is autofluorescence. METHODS: Cells exposed to drug for 24 hrs prior to live cell confocal imaging (N>2).

FIGS. 9A-9B show quantification methods verifying α7-nAChR expression and lead CPP-FITC internalization. 9A&A′. Visualization of α-btx-AF647 labeling α7-nAChR transfected N2a cells. 9B. α-btx-AF647 quantification of α7-nAChR transfected vs non-transfected cells (Students T-test. P<0.01). C. CTCF quantification of lead CPP internalization by α7-nAChRs (One-way ANOVA. P<0.01). METHODS: A&A′) Confocal live cell imaging, 24 hr post-α-btx-AF647 exposure. 9B) Fluorescence detected by Tecan plate reader (mean±SD, N=3). 9C) ImageJ quantification (mean±SD, N=2).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”,“an”, and “the” include plural reference unless the context clearly dictates otherwise. Thus, for example, reference to “a peptide” includes a plurality of such peptides, reference to “the subject” is a reference to one or more subjects and equivalents thereof known to those skilled in the art, and so forth.

By “treat” is meant to administer a peptide, composition, nucleic acid, vector or cell of the invention to a subject, such as a human or other mammal (for example, an animal model), that has an increased susceptibility for developing a disease, or disorder (e.g. inflammation, chronic pain or depression)in order to prevent or delay onset of the disease or disorder, prevent or delay a worsening of the effects of the disease or disorder, or to partially or fully reverse the effects of the disease or disorder. In some aspects, treat can mean to ameliorate a symptom of a disease or disorder (e.g. inflammation, chronic pain or depression).

By “prevent” is meant to minimize the chance that a subject who has an increased susceptibility for developing a disease, or disorder will actually develop the disease, or disorder.

As used herein, the term “subject” or “patient” can be used interchangeably and refer to any organism to which a peptide or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

As used herein, the terms “administering” and “administration” refer to any method of providing a disclosed peptide, composition, nucleic acid, vector or cell of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologous or identical to a reference sequence. In some aspects, a reference sequence can be a portion (e.g. a fragment) of a rabies viral glycoprotein (RVG) nucleic acid sequence or protein sequence (e.g. fragment of RVG). Variants can also include nucleotide sequences that are substantially similar to sequences of peptide disclosed herein. A “variant” can mean a difference in some way from the reference sequence other than just a simple deletion of an N- and/or C-terminal amino acid. Variants can also or alternatively include at least one substitution and/or at least one addition; there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few residues to minimize the alteration of the molecule. However, larger changes may be tolerated in certain circumstances.

Generally, the amino acid or nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 amino acid residue changes as compared to the parent or reference sequence of the invention, and shares or improves biological function, specificity and/or activity of the parent sequence.

Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res. 25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Peptides

Disclosed are peptides that show subtype specific nicotinic acetylcholine receptor (nAChRs) targeting. nAChRs are therapeutic targets for chronic pain, depression, nicotine addiction, and Alzheimer's disease. Rabies viral glycoprotein (RVG) peptides can transport therapeutics across the BBB but have shown low nAChR subtype selectivity. α-neurotoxins, such as α-bungarotoxin, show nAChR subtype selectivity. Disclosed herein are peptides comprising portions (e.g. fragments) of RVG peptides and α-neurotoxins.

Disclosed are peptides comprising a first portion, a second portion and a third portion, wherein the first portion is a nAChR subtype selective peptide, wherein the second portion is a portion of a RVG peptide, and wherein the third portion is a nAChR subtype selective peptide. In some aspects, the nAChR subtype selective peptide can be from an α-neurotoxin.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin. In some aspects, the portion of a RVG peptide is a cell penetrating peptide.

Disclosed are peptides comprising a first portion, a second portion and a third portion, wherein at least the second portion is a portion of a RVG peptide, and wherein at least one of the first or third portions is a nAChR subtype selective peptide In some aspects, the nAChR subtype selective peptide can be from an α-neurotoxin. In some aspects, the portion of the RVG peptide is a cell penetrating peptide.

In some aspects, the disclosed peptides are comprised of fragments of a RVG peptide and fragments of an α-neurotoxin peptide.

In some aspects, the disclosed peptides are capable of >80% inhibition of ACh-mediated currents at 100 μM peptide on no more than two ACh subtypes and <25% inhibition on the other nAChRs. In some aspects, the peptide is capable of >80% inhibition of α7 and α9α10 nAChRs and <25% inhibition of β3α6β2α4β2, α6/α3β2β3, α4β2α5, α3β2, α3β2α3, α4β2, and α3β4. In some aspects, the peptide has an IC₅₀ value in the nM-μM range for α7 and α9α10 receptors while maintaining <25% inhibition on the other subtypes.

### 1. RVG

The disclosed peptides comprise one or more portions or fragments of a RVG peptide. In some aspects, the portion of the RVG peptide is the second portion of the disclosed peptides. In some aspects, the portion of the RVG peptide can be the first and/or third portion of the disclosed peptides in combination with the second portion being a portion of the RVG peptide. In some aspects, the portion of the RVG peptide is not present in the first portion, second portion and third portion at the same time. Thus, at most, only two of the three portions can be from RVG at one time.

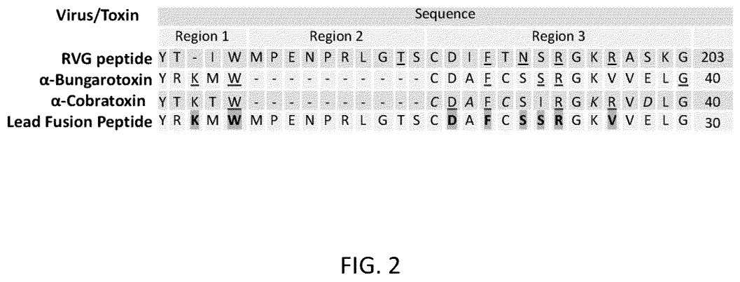

In some aspects, a RVG peptide comprises the amino acid sequence YTIWMPENPRLGTSCDIFTNSRGKRASKG (SEQ ID NO:1), as shown in FIG. 2. In some aspects, a portion of the RVG peptide as described throughout can be any portion of SEQ ID NO:1. For example, in some aspects, the first portion of the disclosed peptides can be YTIW (SEQ ID NO:22). In some aspects, the second portion of the disclosed peptides can be MPENPRLGTS (SEQ ID NO:2). In some aspects, the third portion of the disclosed peptides can be CDIFTNSRGKRASKG (SEQ ID NO:23).

In some aspects, the portion of the RVG peptide comprises the amino acid sequence MPENPRLGTS (SEQ ID NO:2), represented as region 2 in FIG. 2.

In some aspects, the portion of the RVG peptide can be from any strain. For example, the portion of the RVG peptide can be any of those disclosed in Table 1. In some aspects, variants of the portion of the RVG peptide can be a variant of a RVG peptide and can infect different species.

TABLE 1
Example RVG peptides from different strains.

| Toxin/Virus | Sequence | |
|---|---|---|
| Dog Rabies v. | Y T - I W M P E N P R L G T S C D I F T N S R G K R A S K G | 203 |
| (SEQ ID | | |
| NO: 1) | | |
| CVS (Rabies) | Y T - I W M P E N P R P G T P C D I F T N S R G K R A S N G | 203 |
| (SEQ ID | | |
| NO: 3) | | |
| PV (Rabies) | Y T - I W M P E N P R L G M S C D I F T N S R G K R A S K G | 220 |
| (SEQ ID | | |
| NO: 4) | | |
| Sad-B19 | Y T - I W M P E N P R L G M S C D I F T N S R G K R A S K G | |
| (Rabies) | | |
| (SEQ ID | | |
| NO: 4) | | |

In some aspects of the disclosed peptides, not only is the second portion a portion of a RVG peptide, but the first or third portion can also be a portion of a RVG peptide. Thus, as disclosed herein, a peptide having at least 40% identity to one of the disclosed α-neurotoxin peptides can be used for the first and/or third portion of the disclosed peptides. Region 3 of a RVG shown in FIG. 2 has at least 40% identity to CDAFCSSRGKVVELG (SEQ ID NO:9) of the α-bungarotoxin peptide.

2. α-neurotoxin

The disclosed peptides comprise one or more portions or fragments of an α-neurotoxin peptide. In some aspects, the portion of α-neurotoxin peptide is an nAChR subtype selective portion. In some aspects, the α-neurotoxin peptide can be an α-bungarotoxin peptide, for example as shown in FIG. 2. In some aspects, the nAChR subtype selective portion of the α-neurotoxin peptide can comprise the amino acid sequence YRKMWCDAFCSSRGKVVELG (SEQ ID NO:5). In some aspects, the α-neurotoxin peptide can be an α-cobratoxin peptide, for example as shown in FIG. 2. In some aspects, the nAChR subtype selective portion of the α-neurotoxin peptide can comprise the amino acid sequence YTKTWCDAFCSIRGKRVDLG (SEQ ID NO:6).

In some aspects, the nAChR subtype selective portion of the of α-neurotoxin peptide can comprise one or more of the amino acid sequences YRKMW (SEQ ID NO:7, α-bungarotoxin peptide), YTKTW (SEQ ID NO:8, α-cobratoxin peptide), CDAFCSSRGKVVELG (SEQ ID NO:9, α-bungarotoxin peptide), CDAFCSIRGKRVDLG (SEQ ID NO:10, α-cobratoxin peptide). In some aspects the disclosed peptides comprise the amino acid sequence YRKMW (SEQ ID NO:7) and CDAFCSSRGKVVELG (SEQ ID NO:9), both of which are a nAChR subtype selective portion of a α-bungarotoxin peptides. In some aspects the disclosed peptides comprise the amino acid sequence YTKTW (SEQ ID NO:8) and CDAFCSIRGKRVDLG (SEQ ID NO:10), both of which are a nAChR subtype selective portions of a α-cobratoxin peptide. In some aspects, the disclosed peptides comprise the amino acid sequence YTKTW (SEQ ID NO:8) and CDAFCSSRGKVVELG (SEQ ID NO:9). In some aspects, the disclosed peptides comprise the amino acid sequence YRKMW (SEQ ID NO:7) and CDAFCSIRGKRVDLG (SEQ ID NO:10).

In some aspects, the nicotinic acetylcholine receptor (nAChR) subtype selective portion of the α-neurotoxin peptide used in the disclosed peptides can be any portion of those peptides listed in Table 2.

TABLE 2
Examples of RVG sequences.

| | Sequence | | |
|---|---|---|---|
| Virus/Toxin | Region 1 | Region 2 | Region 3 |
| Dog Rabies v. | YT-IW | MPENPRLGTX | CDIFTNSRGKRASKG |
| (SEQ ID NO: 1) | | | (bold G is amino acid |
| | | | 203) |

α7 predominate nAChR selectivity

| | | | |
|---|---|---|---|
| α-Bungarotoxin | YKRKMW | ---------- | CDAFCSSRGKVVELG |
| (SEQ ID NO: 5) | (Y is amino acid 21) | | (bold C is amino acid |
| | | | 30; bold G is amino acid |
| | | | 40) |
| α-Cobratoxin | YTKTW | ---------- | CDAFCSIRGKRVDLG |
| (SEQ ID NO: 6) | (Y is amino acid 21) | | (bold C is amino acid |
| | | | 30; bold G is amino acid |
| | | | 40) |
| Neurotoxin B | YTKTW | ---------- | CDGFCSSRGKRIDLG |
| (SEQ ID NO: 13) | (Y is amino acid 21) | | (bold C is amino acid |
| | | | 30; bold G is amino acid |
| | | | 41) |

α3 predominate nAChR selectivity

| | | | |
|---|---|---|---|
| κ-Bungarotoxin | FLKAQ | ---------- | CDKFCSIRGPVIEQG |
| (SEQ ID NO: 14) | (F is amino acid 21) | | (bold C is amino acid |
| | | | 30; bold G is amino acid |
| | | | 40) |
| κ2-Bungarotoxin | FLKAQ | ---------- | CDKFCPIRGPVIEQG |
| (SEQ ID NO: 15) | (F is amino acid 21) | | (bold C is amino acid |
| | | | 30; bold G is amino acid |
| | | | 40) |
| κ3-Bungarotoxin | FRKAQ | ---------- | CDNFCHSRGPVIEQG |
| (SEQ ID NO: 16) | (F is amino acid 21) | | (bold C is amino acid |
| | | | 30; bold G is amino acid |
| | | | 40 |
| κ-Flavitoxin | FTKAF | ---------- | CDRWCSSRGPVIEQG |
| (SEQ ID NO: 17) | (F is amino acid 21) | | (bold C is amino acid |
| | | | 30; bold G is amino acid |
| | | | 40 |

Heteromeric selective nAChR modulators

| | | | |
|---|---|---|---|
| h. Lynx-1 | MTTR- | ---------- | -TYYTPTRMKV-SKS |
| (SEQ ID NO: 18) | | | |
| h. Lynx-2 | QKEV- | ---------- | -MEQS-AGIMY-RKS |
| (SEQ ID NO: 19) | | | |
| SLURP 1 | MTTL- | ------VTVE | AEYPFNQSPVV-TRS |
| (SEQ ID NO: 20) | | | |
| SLURP 2 | VTTA- | ------TRVL | SNT-EDLPLV-TKM |
| (SEQ ID NO: 21) | | | |

### 3. Combination of RVG and α-Neurotoxin

Disclosed are peptides comprising a combination of the disclosed portion of RVG peptides and nicotinic acetylcholine receptor (nAChR) subtype selective portion of α-neurotoxin peptides. In some aspects, the point of the fusion peptide having a combination of a portion of RVG peptides and nAChR subtype selective portions of α-neurotoxin is to improve the properties, specifically the nAChR subtype selectivity, of RVG. Thus, in some aspects, portions of RVG peptides can be replaced with portions of an α-neurotoxin. In some aspects, neurotoxins have similar sequences to region 1 and region 3 of a RVG peptide as shown in FIG. 2 and Table 2 and therefore those portions of a neurotoxin can replace region 1 and region 3 of the RVG peptide to form the peptides disclosed herein.

Thus, in some aspects, any of the disclosed α-neurotoxin peptides can be combined with the RVG amino acid sequence MPENPRLGTS (SEQ ID NO:2). In some aspects, the RVG sequence is in the middle of two α-neurotoxin peptides. For example, in some aspects, the middle portion of YTIWMPENPRLGTSCDIFTNSRGKRASKG (SEQ ID NO:1), which is the RVG sequence MPENPRLGTS (SEQ ID NO:2), can be inserted in an α-bungarotoxin peptide or a-cobratoxin peptide thus resulting in the peptide YRKMWMPENPRLGTSCDAFCSSRGKVVELG (SEQ ID NO:11) or YTKTWMPENPRLGTSCDAFCSIRGKRVDLG (SEQ ID NO:12), respectively.

Disclosed are peptides comprising a first portion, a second portion and a third portion, wherein the first portion is a nAChR subtype selective portion of an α-neurotoxin, wherein the second portion is a portion of RVG, and wherein the third portion is a nAChR subtype selective portion of an α-neurotoxin, wherein the first portion comprises the amino acid sequence YRKMW (SEQ ID NO:7), YTKTW (SEQ ID NO:8), or YTIW (SEQ ID NO: 22), the second portion comprises the amino acid sequence MPENPRLGTS (SEQ ID NO:2) and the third portion comprises the sequence CDAFCSSRGKVVELG (SEQ ID NO:9), CDAFCSIRGKRVDLG (SEQ ID NO:10), or CDIFTNSRGKRASKG (SEQ ID NO:23).

In some aspects, the combination of a portion of RVG peptide and a nAChR subtype selective portion of an α-neurotoxin can be Region 2 of the RVG peptide shown in FIG. 2 wherein Region 1 and Region 2 of the RVG peptide are replaced with a nAChR subtype selective portion of an α-neurotoxin. In some aspects, the combination of a portion of RVG peptide and a nAChR subtype selective portion of an α-neurotoxin can be Region 1 and Region 2 of the RVG peptide shown in FIG. 2 wherein Region 3 of the RVG peptide is replaced with a nAChR subtype selective portion of an α-neurotoxin. In some aspects, the combination of a portion of RVG peptide and a nAChR subtype selective portion of an α-neurotoxin can be Region 2 and Region 3 of the RVG peptide shown in FIG. 2 wherein Region 1 is replaced with a nAChR subtype selective portion of an α-neurotoxin.

### 4. Variants

Disclosed are variants of any of the disclosed peptides. For example, disclosed are peptides having 40%, 45%, 50%, 55%, 60%, 65%, 70%, 75%, 80%, 85%, 90%, 95%, or 99% identity to any of the peptides disclosed herein.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the first portion comprises an amino acid sequence with 40%, 45%, 50%, 55%, 60%, 65%, 70%, 75%, 80%, 85%, 90%, 95%, or 99% identity to YRKMW (SEQ ID NO:7) or YTKTW (SEQ ID NO:8). In some aspects, the second and third portions can be any of those disclosed herein.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the second portion comprises an amino acid sequence with 40%, 45%, 50%, 55%, 60%, 65%, 70%, 75%, 80%, 85%, 90%, 95%, or 99% identity to MPENPRLGTS (SEQ ID NO:2). In some aspects, the first and third portions can be any of those disclosed herein.

Disclosed are peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin, wherein the third portion comprises an amino acid sequence with 40%, 45%, 50%, 55%, 60%, 65%, 70%, 75%, 80%, 85%, 90%, 95%, or 99% identity to CDAFCSSRGKVVELG (SEQ ID NO:9) or CDAFCSIRGKRVDLG (SEQ ID NO:10). In some aspects, the first and second portions can be any of those disclosed herein.

Disclosed are peptides having 40%, 45%, 50%, 55%, 60%, 65%, 70%, 75%, 80%, 85%, 90%, 95%, or 99% identity to YRKMWMPENPRLGTSCDAFCSSRGKVVELG (SEQ ID NO:11) or YTKTWMPENPRLGTSCDAFCSIRGKRVDLG (SEQ ID NO:12).

In some aspects, the disclosed peptides comprise or consist of the amino acid sequence having one or more amino acid substitutions at positions 3, 5, 14, 17, 19, 22, 23, or 26 of YRKMWMPENPRLGTSCDAFCSSRGKVVELG (SEQ ID NO:11) or YTKTWMPENPRLGTSCDAFCSIRGKRVDLG (SEQ ID NO:12).

In some aspects, variants comprise one or more non-naturally occurring amino acid. In some aspects, non-naturally occurring amino acids can be used to replace any of the amino acids of the sequences described herein. In some aspects, non-naturally occurring amino acids can help prevent peptide breakdown from enzymes within a subject. In some aspects, the non-naturally occurring amino acids do not occur at a residue critical for potency and selectivity.

### 5. Cargo

In some aspects, the disclosed peptides can further comprise a cargo. In some aspects, the cargo can be a therapeutic or molecular tag.

In some aspects, the cargo can be present on the C-terminal or N-terminal end of the disclosed peptides. In some aspects, the cargo can be conjugated to the disclosed peptides via a cleavable linker.

In some aspects, a therapeutic can be siRNA, a viral vector, a nanoparticle, a liposome, a protein, a nucleic acid (DNA or RNA), or a compound. In some aspects, the therapeutic can be anything that treats or ameliorates a symptom of depression or pain. In some aspects, the therapeutic can be a viral vector and the viral vector is adeno-associated viral (AAV) vector. In some aspects, the viral vector is a therapeutic as it carries a gene that can act as a therapeutic.

In some aspects, the cargo can be the addition of a non-naturally occurring amino acid on the C-terminal or N-terminal end of the peptide. In some aspects, the non-naturally occurring amino acid is a non-naturally occurring arginine. In some aspects, the non-naturally occurring amino acid can act as a linker. For example, a non-naturally occurring amino acid can act as a linker for attaching siRNA to the disclosed peptides. In some aspects, the presence of non-naturally occurring amino acids can help prevent peptide breakdown from enzymes within a subject.

In some aspects, the cargo is a molecular tag and the molecular tag can be a labeling agent. For example, a labeling agent can be a fluorescent molecule (e.g., fluorescent compound or fluorescent protein) or a dye.

In some aspects, the cargo is attached to the peptide via a linker. In some aspects, the cargo can be a therapeutic or imaging agent. In some aspects, a therapeutic can be a protein, nucleic acid, or compound. In some aspects, a therapeutic can be, but is not limited to, siRNA, a viral vector, a nanoparticle, a liposome, a protein, or a compound. In some aspects, the imaging agent can be, but is not limited to, a fluorescent agent. In some aspects, there is a linker that joins one of the disclosed peptides and the cargo. In some aspects, the linker can be, but is not limited to, PEG, aminohexanoic acid, glycines, serines, D-Arg, sulfides (e.g. disulfide bond to cyclize the peptide), beta-alanine, (2-aminoethoxy) acetic acid, 6-aminohexanoic acid, or 8-amino-3,6-dioxaoctanoic acid. In some aspects, the linkers can be flexible, rigid and/or cleavable. In some aspects, the linker is on the C-terminal end of the peptide. In some aspects, the linker is on the N-terminal end of the peptide.

Any suitable linker can be used in accordance with the present invention. The peptide linkages can be selected from the group consisting of: —CH₂NH—, —CH₂S—, —CH₂—CH₂—, —CH═CH— (cis and trans), —COCH₂—, —CH(OH)CH₂—, —CH₂SO—, etc. by methods known in the art and further described in the following references: Spatola (1983) p. 267 in Chemistry and Biochemistry of Amino Acids, Peptides, and Proteins, B. Weinstein, eds., Marcel Dekker, New York; Spatola (1983) Vega Data 1(3) Peptide Backbone Modifications. (general review); Morley (1980) Trends Pharm Sci pp. 463-468 (general review); Hudson et al. (1979) Int J PeptProt Res 14:177-185 (—CH₂NH—, CH₂CH₂—); Spatola et al. (1986) Life Sci 38:1243-1249 (—CH₂—S); Hann, (1982) J ChemSoc Perkin Trans I 307-314 (—CH—CH—, cis and trans); Almquist et al. (1980) J Med. Chem. 23:1392-1398 (—COCH₂—); Jennings-White et al. (1982) Tetrahedron Lett. 23:2533 (—COCH₂—); Szelke et al., European Appln. EP 45665 (1982) CA: 97:39405 (1982) (—CH(OH)CH₂—); Holladay et al. (1983) Tetrahedron Lett 24:4401-4404 (—C(OH)CH₂—); and Hruby (1982) Life Sci., 31:189-199 (—CH₂—S—)).

One particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), reduced antigenicity, and others.

In one aspect, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g., esterase cleavable linker), ultrasound-sensitive linkers, x-ray cleavable linkers, etc.

### C. Nucleic Acids

Disclosed are nucleic acid sequences capable of encoding one or more of the disclosed peptides. For example, in some aspects, a nucleic acid sequence capable of encoding one of the disclosed peptides comprises the sequence

(SEQ ID NO: 24)
TACAGAAAGATGTGGATGCCCGAGAACCCCAGACTGGGCACCAGCTGCG
ACGCCTTCTGCAGCAGCAGAGGCAAGGTGGTGGAGCTGGGCTGA

As this specification discusses various peptide sequences it is understood that the nucleic acids that can encode those peptide sequences are also disclosed. This would include all degenerate sequences related to a specific polypeptide sequence, i.e. all nucleic acids having a sequence that encodes one particular peptide sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed peptide sequences.

### D. Vectors

Disclosed are vectors comprising one or more of the nucleic acid sequences disclosed herein. In some aspects, the vectors can be used as a gene therapy tool.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector (AAV). In some aspects, the AAV can be AAV9. In some aspects, the vector can be a non-viral vector, such as a DNA based vector.

In some aspects, the vector is AAV9. In some aspects, a benefit of using AAV vectors can be that AAV is non-pathogenic in human and elicits a very mild immune response. In some aspects, the disclosed vectors are considered recombinant vectors. A recombinant AAV can lack two essential genes for viral integration and replication but remains primarily episomal and can persist in non-dividing cells for long periods of time. The tissue specificity of AAV can be determined by the viral capsid serotype, which allows targeting the gene of interest to specific tissues. In some aspects, AAV9 can be used to efficiently target the heart, although it also can target to other organs/tissues such as muscles, lungs, kidneys.

### 1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence encoding a peptide consisting of 30 to 50 amino acids, wherein the 30 to 50 amino acids comprise an amino acid sequence of YECDIPIGAGICASYQTQTNSPRRARSVAS (SEQ ID NO:1), YECDIPIGAGICASYQTQTNSRRRARSVAS (SEQ ID NO: 2) or a variant of SEQ ID NO:1 or SEQ ID NO:2.

The “control elements” present in an expression vector are those non-translated regions of the vector—enhancers, promoters (e.g. a human pro-B-type natriuretic protein (hBNP) promoter), 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes B-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage

### E. Cells

Disclosed are cells comprising one or more of the peptides, nucleic acid sequences or vectors disclosed herein.

In some aspects, the cells are mammalian cells. In some aspects, the cells are human cells.

In some aspects, the cells are primary cells. In some aspects, the cells are a cell line.

### F. Compositions

Disclosed herein are compositions comprising any of the disclosed peptides, nucleic acids, vectors or cells. For example, disclosed are compositions comprising a peptide comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin.

Disclosed are compositions comprising one of the disclosed peptides, wherein the composition further comprises a cargo. In some aspects, the cargo is attached to the peptide via a linker as described above.

Also disclosed herein are pharmaceutical compositions comprising the disclosed peptides, nucleic acids vectors, or cells. Disclosed are pharmaceutical compositions comprising peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin.

Thus, in some aspects, the disclosed compositions can further comprise a pharmaceutically acceptable carrier. For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. AR. Gennaro, Mack Publishing Company, Easton, P A 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about α7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, glucose and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like.

### 1. Delivery of Compositions

In the methods described herein, delivery (or administration) of the disclosed peptides, compositions, nucleic acids, cells or vectors disclosed herein to cells or a subject can be via a variety of mechanisms. As defined above, disclosed herein are compositions comprising any one or more of the disclosed peptides comprising a first portion, a second portion and a third portion, wherein the first portion is a nAChR subtype selective portion of an α-neurotoxin, wherein the second portion is a portion of a RVG peptide, and wherein the third portion is a nAChR subtype selective portion of an α-neurotoxin and can also include a carrier such as a pharmaceutically acceptable carrier. For example, disclosed are pharmaceutical compositions, comprising a peptide comprising the sequence of YRKMWMPENPRLGTSCDAFCSSRGKVVELG (SEQ ID NO:11) or YTKTWMPENPRLGTSCDAFCSIRGKRVDLG (SEQ ID NO:12) and a pharmaceutically acceptable carrier.

The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, glucose, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed peptides, nucleic acids, vectors or cells.

In some aspects, the disclosed compositions, peptides, nucleic acids, vectors, or cells are administered in combination with one or more additional agents. In some aspects, the additional agent can be, but is not limited to, a traditional therapeutic for the disease or disorder being treated. For example, a traditional therapeutic can be, but is not limited to, a therapeutic that treats chronic pain, depression, nicotine addiction, and Alzheimer's disease.

### G. Methods

Disclosed are methods of using any of the disclosed peptides as a therapeutic. For example, the disclosed peptides alone, without an additional therapeutic attached, can be used for therapeutic purposes. Furthermore, in some aspects, attaching a therapeutic to the disclosed peptides can provide a further therapeutic advantage.

α9α10 and α7 nAChRs play a role in chronic pain and depression. In some aspects, many of the disclosed methods occur when the disclosed peptides inhibit or desensitize α9α10 and α7 nAChRs In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion.

Disclosed are methods of modulating an α7 and/or α9α10 nAChR response in a subject comprising administering any of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. In some aspects, modulating can involve an agonist, negative modulation, positive modulation, and/or a co-agonist.

Disclosed are methods of antagonizing an α7 and/or α9α10 nAChR response in a subject comprising administering any of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. Disclosed are methods of antagonizing an α7 and/or α9α10 nAChR response in a subject comprising administering a peptide comprising a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof.

Disclosed are methods of inhibiting α7 and/or α9α10 nAChR in a subject comprising administering any of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. Disclosed are methods of inhibiting α7 and/or α9α10 nAChR in a subject comprising administering a peptide comprising a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof.

Disclosed are methods of decreasing inflammation in a subject comprising administering any of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. Disclosed are methods of decreasing inflammation in a subject comprising administering a peptide comprising a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof. In some aspects, a9 and a10 nAChR mRNA transcripts are present in immune cells including monocytes, macrophages, mast cells, B cells, and T lymphocytes. Therefore, in some aspects, antagonizing α9α10 nAChRs can reduce inflammation.

Disclosed are methods of treating or ameliorating a symptom of chronic pain in a subject comprising administering a composition comprising one or more of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. Disclosed are methods of treating or ameliorating a symptom of chronic pain in a subject comprising administering a peptide comprising a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof. In some aspects, the disclosed peptides can act as a dual therapy as it can 1) antagonize specific nAChR subtypes assisting with pain symptoms, and/or 2) deliver a therapeutic into a cell. In some aspects, hypercholinergic tone, in part caused by α9α10 nAChR activation, is associated with neuropathic pain, and pharmacologically desensitizing a9a10-nAChRs improves symptoms in mammals (including pain). In some aspects, the delivery of a therapeutic into a cell can be achieved through the disclosed peptides aiding in cell penetration. In some aspects, a therapeutic can be Conotoxin RgIA which is useful in the management of neuropathic and inflammatory pain via inhibition of α9α10 nAChRs. In some aspects, a therapeutic that mimicks the functional effects of RgIA can be used to treat neuropathic pain.

Disclosed are methods of treating or ameliorating a symptom of depression in a subject comprising administering a composition comprising one or more of the disclosed peptides to a subject in need thereof. In some aspects, the peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin. In some aspects, the RVG peptide is a cell penetrating portion. Disclosed are methods of treating or ameliorating a symptom of depression in a subject comprising administering a peptide comprising a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof. In some aspects, the disclosed peptides can act as a dual therapy as it can 1) antagonize specific nAChR subtypes assisting with depression and pain symptoms, and/or 2) deliver a therapeutic into a cell. In some aspects, hypercholinergic tone, in part caused by α7-nAChR activation, is associated with depression, and pharmacologically desensitizing α7-nAChRs improves depressive symptoms. In some aspects, a therapeutic that promotes brain-derived neurotrophic factor (BDNF) production can be used. By reducing expression of programmed cell death 4 (Pdcd4) protein by small interfering RNA (siRNA) cargo delivered by a disclosed peptide, BDNF expression is enhanced and reverses depressive-like behaviors in mice. In some aspects, siRNAs can reduce protein expression of proteins are involved in depression, however, siRNAs can be rapidly broken down in the body and have limited success crossing the blood brain barrier unassisted, restricting their therapeutic potential. In some aspects, to protect siRNAs from degradation, siRNAs can be conjugated to one or more of the disclosed peptides, which can facilitate CNS siRNA delivery.

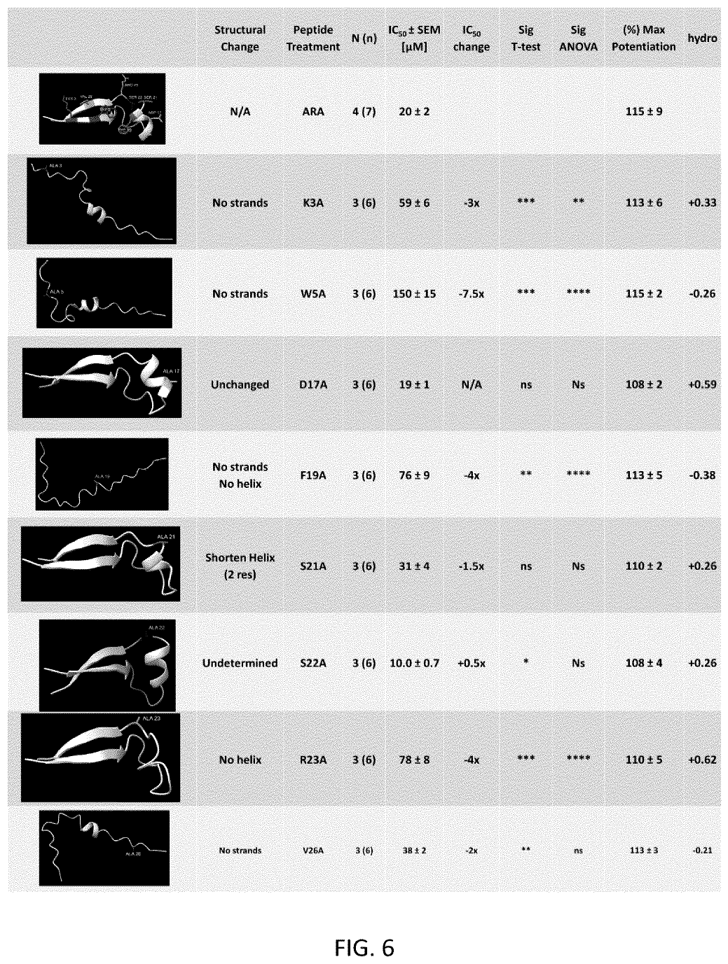

Disclosed are methods of increasing nAChR subtype specificity comprising administering a composition comprising one of the disclosed peptides. Disclosed are methods of increasing nAChR subtype specificity comprising administering a composition comprising a peptide comprises a first portion, a second portion and a third portion, wherein the first portion is a nAChR subtype selective portion of an α-neurotoxin, wherein the second portion is a portion of a rabies viral glycoprotein (RVG) peptide, and wherein the third portion is a nAChR subtype selective portion of an α-neurotoxin to a subject in need thereof. In some aspects, the RVG peptide is a cell penetrating portion. Disclosed are methods of increasing nAChR subtype specificity comprising administering a composition comprising one of the disclosed peptides, wherein the peptide comprises or consists of the amino acid sequence having one or more amino acid substitutions at positions 3, 5, 14, 17, 19, 22, 23, or 26 of YRKMWMPENPRLGTSCDAFCSSRGKVVELG (SEQ ID NO:11), to a cell. In some aspects, the cell can be in vitro or in vivo. K3, W5, T14, F19, S22, R23, V26, of SEQ ID NO:11, have been identified as residues that are important contributors for potency. In some aspects, residues Y1, K3, W5, F19, S21, S22, R23, G24, K25, V26, E28, and G30 can improve potency and/or specificity. In some aspects, the mutations present in the beta-sheets can disrupt the structure of the peptide. In some aspects, the residues in the flexible loop areas can be involved in potency. FIG. 6 shows examples of the predicted structures of the parent peptide (row 1) and residues that have been alanine mutated are shown in stick view. α7 nAChR functional peptide potency and maximum potentiation are shown for statistical comparison for subsequent peptides that have been mutated at the indicated positions (3, 5, 17, 19, 21, 22, 23, and 26). FIG. 6 shows examples of the predicted structural outcomes of a single alanine mutation at the indicated position, and the functional effects on α7 nAChR potency and potentiation.

In some aspects, a subject in need thereof has central nervous system (CNS) hypercholinergic tone. In some aspects, a subject having CNS hypercholinergic tone has chronic pain or depression. In some aspects, hypercholinergic tone is excessive (above normal) cholinergic signaling. In some aspects, a subject in need thereof has nicotine addiction.

In some aspects, the disclosed compositions can be used as a research tool. Thus, disclosed are methods of detecting nAChRs or cells having nAChRs comprising administering a composition comprising one of the disclosed peptides to a cell or cell population. In some aspects, the administration is in vivo. In some aspects, the administration is in vitro.

### H. Dosages

Disclosed are dosing regimens comprising administering a single dose of one or more of the disclosed compositions or peptides to a subject in need thereof, wherein the single dose comprises an amount effective to modulate a specific subtype of nAChR or to treat a particular disorder, such as pain or depression.

Disclosed are dosing regimens comprising administering at least two doses of one or more of the disclosed compositions or peptides to a subject in need thereof, wherein each dose is the same concentration. In some aspects, each dose after a first dose can be decreased. In some aspects, each dose after a first dose can be increased.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising one or more of the disclosed peptides, nucleic acid sequences, vectors, cells or compositions. The kits also can contain a therapeutic and/or a linker to join a peptide to a therapeutic.

### Examples

### A. Example 1: Treatment of Pain and Depression

This study has designed novel fusion peptides derived from the RVG and α-bungarotoxin (a-btx) to generate a lead peptide that functionally inhibits α9α10 and α7 nAChRs, while minimally targeting nine other nAChR subtypes. In this work improvements upon the generated lead peptide are made to solve the critical need for novel, non-addictive treatments for pain and depression that move the field away from opioid dependency.

Provided are methods to develop nAChR subtype selective peptides for the treatment of pain and depression. These methods seek to: 1) Improve the potency and selectivity of the lead peptide by mutagenesis of predetermined residues. 2) Determine the cytotoxicity profile of the lead peptide and future analogues. Through mutagenesis, hot spot residues have been identified that reduce the peptide's potency, and substitutions that improve potency for α7 nAChRs. Generation of nAChR subtype selective RVG-neurotoxin fusion peptides, in addition to modifying nAChR activation, can also effectively deliver therapeutics into cells expressing specific nAChR subtypes, as accomplished with non-selective RVG CPPs.

Provided are methods to: 1) Improve the lead RVG fusion peptide potency and selectivity using mutagenesis. Based on data and literature, there are eight key residues that are important in altering peptide potency and selectivity. Two-electrode voltage clamp (TEVC) electrophysiology can be used to determine the functional potency and selectivity of the mutated fusion peptides with nAChR subtypes; and 2) Define lead RVG fusion peptide cytotoxicity using cell lines expressing α7 or α9α10 nAChRs. Cell viability can be assessed by adding a fluorescent cell viability reagent and using a microplate reader. A concentration range that is non-cytotoxic can be established.

The potency and selectivity of the lead peptide can be improved, in addition to determining the potential cytotoxicity effects. The disclosed peptides can be used to correct aberrations in conditions associated with hypercholinergic activity such as chronic neurological pain and depression.

### 1. Significance

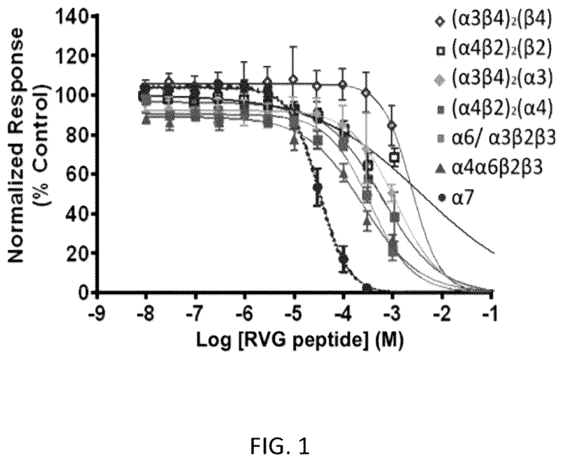

The use of peptides as therapies has increased yearly due to augmented specificity that is achievable with peptides versus small molecules. nAChRs are therapeutic targets for chronic pain, depression, nicotine addiction, and Alzheimer's disease. Snake toxins and conotoxins are proteins capable of discerning between nAChR subtypes, but are cytotoxic, lack stability, and are not readily bioavailable. RVG peptides have been used successfully to deliver therapeutics into the mouse brain by a proposed nAChR-mediated mechanism. However, data demonstrates that the RVG peptide is not target specific (FIG. 1).

The RVG contains a neurotoxin-like region with significant sequence homology to snake α-neurotoxins, which function as potent nAChR subtype selective antagonists. Using regions of α-neurotoxins that facilitate subtype specificity, in combination with the RVG peptide, could generate potent, nAChR subtype specific, fusion peptides. Resulting peptides would be beneficial in research studying nAChRs in biological organisms. RVG fusion peptides could also be useful in the clinical setting to target specific nAChR subtypes to alter receptor behavior. Critically, ligands and peptides, which antagonize α7 and α9α10 nAChRs, have proven to be useful in neurological pain and chronic depression treatments. These strategies may provide an alternative to addictive opioid treatments and can increase a patient's risk for developing depression.

The lead RVG fusion peptide encompasses residues from RVG and α-btx loop II (FIG. 2). To improve lead peptide potency (specific aim 1), predetermined residues can be mutated. α-neurotoxin loop II residues identified as key sites for mutation modify ligand specificity to nAChR subtypes, usually reducing potency (FIG. 2). Previously tested RVG CPPs were not cytotoxic at the concentrations used, however, cytotoxicity profiles were not specifically evaluated. Cytotoxicity of the lead peptide can be evaluated in cell lines transfected with nAChR subtypes (specific aim 2). The lead peptide is novel in that it antagonizes acetylcholine (ACh)-mediated α7 and α9α10 nAChR responses, making it a potentially innovative treatment strategy for chronic neurological pain and depression. The lead peptide utilizes an existing high affinity neurotoxin and an RVG penetrating peptide as scaffolds to generate novel peptides. These peptides have high therapeutic potential and can be CPPs.

As the lead RVG fusion peptide functionally inhibits α7 and α9α10 nAChRs, conditions characterized by cholinergic hyperactivity, such as depression and chronic neurological pain are likely to be the best disease candidates. Involvement of the cholinergic system in depression is supported by the increased rate of smoking among depressive persons, and the ability of the nicotine patch to reduce depressive symptoms. Chronic low levels of nicotine desensitize, rather than stimulate, nAChRs, indicating that diminished nAChR function may be important for the anti-depressive effects. Further, molecules that promote the desensitized state of α7 nAChRs have been verified as beneficial in depression treatments.

α7 nAChRs are abundantly expressed in the CNS, and are involved in disorders of hypercholinergic activity including depression. α9α10 nAChRs are not expressed in the adult brain, but are expressed in the PNS, and in immune cells where they modulate neuroinflammation. Inflammation and neuropathic pain can be improved by antagonizing α9α10 nAChRs. As depression and pain are often co-morbid, having one ligand that can improve both conditions is likely to be extremely valuable. The application of RVG fusion peptides can be applicable to other diseases where specific nAChR subtypes are overexpressed, or hypercholinergic activity occurs. The lead RVG fusion peptide has promise to remedy these treatment gaps.

i. Aim 1

Safe and successful delivery of therapeutics via RVG CPPs into the murine CNS has been accomplished by a putative nAChR-mediated mechanism. RVG CPPs deliver cargo into murine brain regions (e.g. cortex, striatum, thalamus), which express α4β2, α7, and α6β2β3, among other subtypes. These studies provide evidence that RVG peptides may not be nAChR subtype selective, a conclusion supported by our preliminary data (FIG. 1).

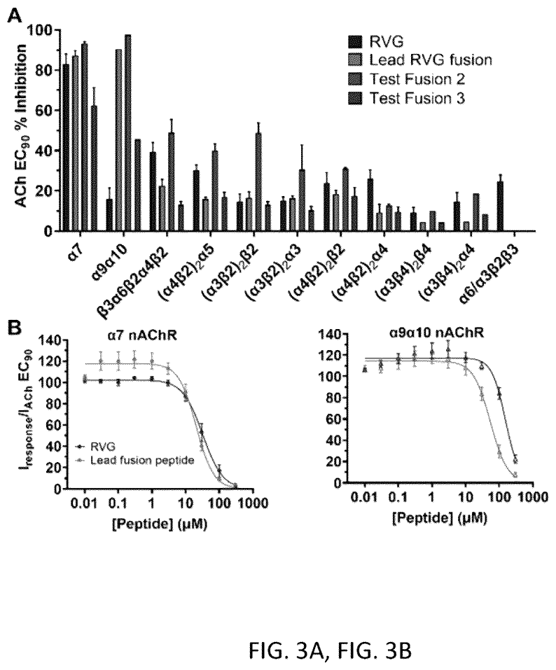

Given that the RVG neurotoxin-like region has high sequence homology with snake α-neurotoxins (FIG. 2), a series of RVG fusion peptides were developed and analyzed using a rapid screening protocol. A positive hit is classified as a peptide that shows >80% inhibition of ACh-mediated currents at 100 μM peptide on no more than two subtypes, and <25% inhibition on the other nAChRs. The parent RVG peptide demonstrated a preference for the α7 nAChR (>80% inhibition) but shows >25% inhibition on at least four other nAChR subtypes (FIG. 3A). The lead peptide has high selectivity and potency for α7 and α9α10 nAChRs, and <25% inhibition of other tested subtypes (FIG. 3). However, these properties can be improved so that the final optimized peptide has nM potency and a wider selectivity window.

To improve the potency of the lead peptide, clues identifying α-neurotoxin residues, or ‘hot spots’, important for ligand affinity and subtype specificity can be found in the literature. An α7 nAChR bound to α-btx cryo-EM structure shows loop II penetrating deeply into the acetylcholine (ACh) binding pocket. At the loop II tip, Phe32 and Arg36 form cation-π interactions with α7 nAChR Tyr187, and α-btx Ser35 interacts electrostatically with Glu116. Mutating α-btx residues Lys23, Trp25, Phe29, Arg33, and Gly40 reduced binding affinity for Torpedo AChR, and mutating similar RVG peptide residues showed analogous affects. α-cobratoxin Trp25, Asp27, Phe29, Arg33, and Arg36 are implicated in the binding of muscle and α7 nAChRs. While Lys23 appears to be only important for muscle subtype binding, Ala28, Cys26-Cys30, and Lys35 are important for α7 nAChR. These studies highlight that there is a positively charged cluster surrounded by hydrophobic side chains in α-neurotoxins that appear critical for specific nAChR interactions.

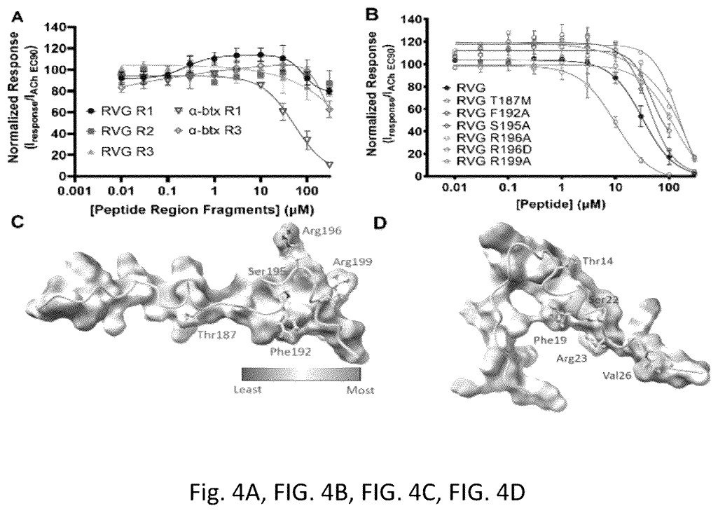

Little work has pinpointed RVG residues that interact with nAChRs. Molecular modeling of the Asn194-Ser195-Arg196-Gly197 tetrapeptide identified these residues form an essential part of the binding site, and Asn and Arg demonstrate ACh molecular mimicry. Our preliminary data shows RVG Region 1 and 2 to have minimal effects on α7 nAChR mediated currents, while Region 3 slightly antagonizes (FIG. 4A). However, α-btx Region 1 and Region 3 inhibit α7 nAChRs. RVG Region 2 does not contribute to potency, but may prove to be important for CPP potential as an Asn194Lys exchange enhances rabies virus cell penetration [54]. These data reveal that several regions are necessary to make an efficacious peptide.

In efforts to identify hot spot residues, our preliminary data show that mutating RVG residues Arg196, Arg199, and Phe192 to Ala significantly weakened potency (FIG. 4B). Interestingly Ser195Ala mutation had minimal effects on α7 nAChR potency. Further investigations into side chain properties at position Arg196, revealed that Lys (charge maintained, size decreased) or Asp (size maintained, charge swap) decreased potency 3- and 7-fold, respectively (FIG. 4B). An RVG Thr197Met mutation, found in the Pasture rabies virus, improved potency by 8-fold (FIG. 4B). These data reveal that mutating residues important in α-neurotoxin potency also alter RVG peptide potency, highlighting functional similarities. This type of protein-receptor structure information provides insight into interactions that confer toxin affinity, and are useful in predicting which peptide residues can alter nAChR potency.

ii. Aim 2

Few studies have addressed the cytotoxicity profiles of RVG-derived CPPs. Some studies have found CPPs to be cytotoxic, especially chimeric peptides, which can be useful to kill certain cells, such as cancerous cells. The lead fusion peptide contains segments of loop II from α-btx and thus this study can define the cytotoxicity profile.

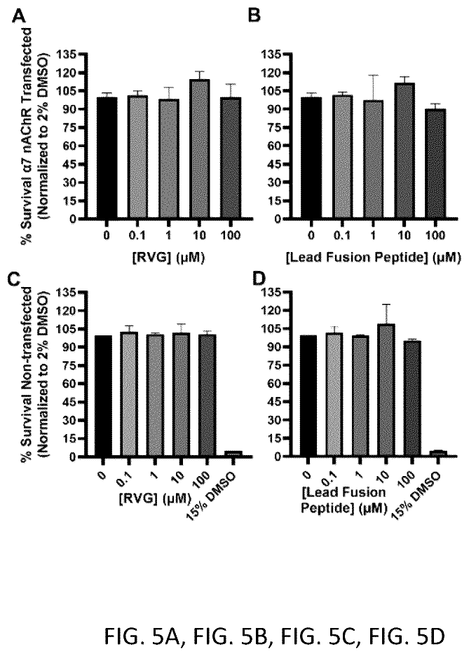

α-btx binds postsynaptic muscle, α7, and α9α10 nAChRs. Blocking cholinergic receptors with α-btx at neuromuscular junctions paralyzes skeletal muscles, which can be lethal. The lead fusion peptide uses small regions of α-btx, reducing, but not eliminating, the risk of cytotoxicity. To test lead fusion peptide cytotoxicity, the alamarBlue HS Cell Viability Reagent assay (ThermoFisher Scientific, Waltham, MA) can be used. Importantly, the data shows that parent and lead fusion peptides appear not to be cytotoxic 24h post incubation using α7 nAChR transfected and non-transfected N2a cells (FIG. 5). Incubation with 15% DMSO kills cells, demonstrating we can detect changes in viability (FIGS. 5C and 5D). Peptides that kill <15% of cells at 100 μM can be considered successful.

### 2. Innovation

Devising fusion peptides that utilize existing high affinity toxins as a scaffold is a new avenue for increasing the utility of RVG peptides as CPPs with cell-type specificity. The therapeutic potential is convincing as RVG peptides cross the BBB to deliver cargo into the mouse brain. The status quo to using RVG CPPs to deliver CNS therapeutics assumes that RVG CPPs target α-btx sensitive nAChRs. Importantly, studies demonstrate RVG CPPs are not nAChR subtype selective due to this peptide's penetrating affects in a variety of neuronal cell lines and brain regions. These studies, and this data, demonstrate that the common problem of CPPs lacking cell and tissue specificity are also true for current RVG CPPs, hindering therapeutic potential. Reducing off-target interactions of RVG peptides are likely to minimize potential side effects and improve the safety profile. Refinement of the novel lead fusion peptide will overcome this barrier by improving nAChR subtype specificity and potency in efforts to reduce off-target and cytotoxic effects.

Developing nAChR subtype selective ligands that could assist in targeted delivery of tagged liposomes and adeno-associated virus' (AAVs). These studies can have significant translational importance in developing improved antidepressants and analgesic. Chronic pain patients tend to have comorbid depression symptoms. Ligands that inactivate α7 and α9α10 nAChRs have been shown to be successful in treating individuals in clinical settings. A refined and potent RVG fusion peptide that antagonizes both α7 and α9α10 nAChRs with high potency and selectivity, while not being cytotoxic can be extremely beneficial in the clinical setting, even if the optimized fusion peptide is not a CPP.

### 3. Approach

i. Specific Aim 1: Improve Our Lead RVG Fusion Peptide Potency and Selectivity Using Mutagenesis.

Provided are methods to improve the lead RVG fusion peptide potency and selectivity by determining key residues for α7 and α9α10 nAChR interactions. Mutating residues that have been identified to modify nAChR potency or subtype selectivity, either in the parent RVG peptide or in intact α-neurotoxins, can improve the lead peptide potency and selectivity. The approach is to create and evaluate these analogues for changes in nAChR potency and selectivity.

a. Research Design

Provided are methods to prepare novel peptides that are subtype selective with high potency, Ala screening can be performed on eight lead fusion peptide residues in Regions 1 and 2 that have been shown to be important for α-neurotoxins or RVG and α7 nAChR interactions: Lys3, Trp5, Asp17, Phe19, S21, S22, Arg23, and Val26 (FIG. 2). All mutations can be modeled prior to synthesis using ChimeraX1.3 and AlphaFold to predict potential structural and physiochemical changes. As shown in FIGS. 4C and 4D, parent RVG and lead fusion peptides are predicted to have distinct structures indicating that different residues can modulate nAChR interactions. For identified hot spots, additional mutations can occur to alter side chain physiochemical properties in efforts to improve potency and selectivity. A fully optimized RVG fusion peptide can contain multiple mutated residues. Derivates of the lead RVG-neurotoxin peptide with nM potency for α7 and α9α10 nAChRs, while antagonizing other nAChR subtypes <25% can be considered successful and cytotoxicity profiles can be evaluated (Specific Aim 2).

Functional expression of nAChR subtypes: Using two-electrode voltage clamp (TEVC) electrophysiology, the RVG fusion peptides are evaluated on nAChR subtypes. nAChRs are pentameric and are composed of a(2-10) and f(2-4) subunits. The α4β2, α7, a3β2, α3β4, α4α6β2β3, α6β2β3, and α4α5β2 subtypes can be studied due to the role of these receptors modulating CNS function, and are expressed in brain regions involved in depression (e.g. striatum, thalamus, hypothalamus, amygdala). Peripheral α9α10 and muscle subtypes can be tested as RVG and α-btx interact with these subtypes, and α9α10 modulates neuropathic pain.

Human nAChRs can be expressed in Xenopus laevis oocytes using cRNA microinjections. Isoforms that contain two a subunits and three β subunits can be achieved using a 1:30α:β cRNA injection ratio, or in a 30:1α:β cRNA injection ratio for isoforms with three a and two 0 subunits. To increase a6 subunit expression, an α6 (N-terminal) and 3 (C-terminal) chimera (α6/α3) can be used. Expression of the α6/α3β2β3 subtype can be achieved by injecting a 2:2:1 ratio of the α6/α3:β2:β3 subunits. Expression of the α4α6β2β3 (stoichiometry ((α6)₁(α4)₁(β2)₂(β3)₁) subtype can be achieved using a subunit-linked construct. The adult muscle subtype can be expressed using a cRNA injection ratio of 2:1:1:1 of α₁:β₁:δ:ε subunits. Injected oocytes can be incubated for 2-6d, a period where isoform shifts have not been observed, prior to TEVC recording. Oocytes can be purchased from IACUC certified EcoCyte Biosciences (Austin, TX). Experiments can be conducted on >2 cRNA synthesis batches and ≥4 oocyte isolations from unique individuals (N).

ACh concentration response curves will be generated by applying a range of concentrations (0.001-30,000 μM, nAChR subtype dependent) for is followed by a 91s wash using Oocyte Ringers 2 (OR2) recording buffer. In all recording solutions, atropine sulfate (1.5 μM) can be added to block potential endogenous muscarinic responses, and 0.1% BSA to prevent peptide adherence to plastics. A range of BSA concentrations was tested and identified 0.1% BSA to have no effect on oocyte viability or ACh mediated response profiles. Response data can be normalized to the ACh concentration that produced the greatest amount of function for a given nAChR subtype and can be used to determine ACh potency. Concentration response profiles can be fit using GraphPad Prism 9.3 Software (San Diego, CA) non-linear curve fitting with standard built-in algorithms. Data can be analyzed using Student's t-test to compare pairs of groups or by one-way ANOVA and Tukey's multiple comparison tests to evaluate the means of three or more groups. The determined half maximal effective concentration (EC₅₀) compares with literature values ensuring that we are expressing the desired receptor (data not shown). The ACh EC₉₀ concentration can be used to test the potency of the RVG peptide.

Functional evaluation of RVG-neurotoxin fusion peptide derivatives on nAChRs: To determine potency of the RVG fusion peptides, concentration-response data can be collected by pre-incubating nAChR expressing oocytes with increasing concentrations of peptide (0.01-300 μM) for 30s before is application of the ACh EC₉₀ concentration on α7 and α9α10 nAChRs. The fusion peptide modified ACh-induced currents can be measured and compared to the ACh EC₉₀ response prior to application of the tested peptides (FIG. 1 and FIG. 3). A 225s wash time will be applied in between drug applications. The half maximal inhibitory concentration (IC₅₀) and n_{H} values can be determined for each test peptide. Peptides can be purchased from ELIM Biopharmaceuticals, Inc. (Hayward, CA) and stored lyophilized in the dark under −20° C. conditions.

For peptides that show an improvement in potency (nM range), subtype selectivity on 12 nAChRs can be evaluated quickly and cost effectively using our rapid screening procedure. Peptides can be tested for subtype selectivity by pre-applying (30s) of a high concentration of peptide (100 μM), followed by a is ACh (EC₉₀) application. All peptide responses can be compared to those of ACh (EC₉₀) when applied in absence of the peptide. It is anticipated that fusion peptides can inhibit ACh-induced currents, as seen for the parent RVG peptide (FIGS. 1 and 3), but other modes of activity may be observed. Peptides that have IC₅₀ values in the nM range for α7 and α9α10 receptors while maintaining <25% inhibition on the remaining subtypes will progress and be evaluated for cytotoxicity effects. An α priori power analysis was conducted using R 4.0.5 for sample size estimation, based on preliminary data (N=2), which compared the RVG peptide to the lead peptide. The preliminary data effect size was 10, with a significance criterion of a=0.05 and power=0.95. The minimum sample size needed with this effect size is N=3 for a paired Student's t-test. Thus, a sample size of N>4 is adequate.

ii. Define Lead RVG Fusion Peptide Cytotoxicity Using Cell Lines Expressing α7 or α9α10 nAChRs.

Provided are methods to determine if the lead RVG fusion peptide is cytotoxic to neuronal and peripheral cells. Optimized RVG fusion peptide(s) can also be tested. The approach is to treat nAChR transfected cells with increasing concentrations of peptide for 24-72 hr and measure cell viability.

a. Research Design:

The lead RVG fusion peptide can be evaluated for its impact on cell health using neuronal-like N2a and peripheral-like HEK cell lines (American Type Culture Collection (ATCC), Manassas, VA). To express nAChRs, N2a cells can be transiently transfected using protocol adopted from. α7 nAChRs can express poorly and need to be co-transfected with the chaperone proteins RIC-3 or NACHO. Cells can be cultured and maintained in 50% Dulbecco's Modified Eagle's Medium (DMEM)/50% Opti-MEM supplemented with 10% fetal bovine serum (VWR, Radnor, PA), and incubation conditions of 37° C. and 5% CO₂. To transfect cells, 0.5-5 μg of each nAChR subunit plasmid DNA can be mixed with cationic lipids (based on manufacturer suggestions) and the Lipofectamine 2000 reagent (ThermoFisher Scientific) followed by an 20 min incubation at room temperature. The transfection media can be added to the cells followed by 24h of incubation. Cells can be rinsed and the lead peptide can be added at concentrations initially around the IC₅₀ concentration for 24 and 72 hrs. As a positive control, 15% DMSO can be used to induce maximal cell death (FIG. 4). To determine cell viability, 1/10^{th} volume of the alamarBlue HS Cell Viability Reagent (ThermoFisher Scientific) can be added and cells can be incubated for 2-4 hrs. Upon entering healthy cells, the resazurine-based reagent is reduced to resorufin, a process that is not possible in dead cells. After incubation, fluorescence values can be determined using the Tecan Spark microplate reader (Tecan, Switzerland). Florescence changes allow us to determine the cytotoxic effects of RVG fusion peptides (FIG. 5). Samples can be run in duplicate per plate to ensure reproducibility. Experiments will be replicated in untransfected cells. Protocols can be modified as needed for HEK cells. Each cytotoxicity assay can be performed on >4 separate transfections.

### B. Example 2: Treating Depression

Delivery of siRNA or drug-carrying nanoparticles into the CNS by peripheral injection is possible with use of cell penetrating peptides (CPP), however current CPPs are not selective for specific cellular macromolecules limiting their use. The non-neurotoxic lead CPP is highly selective for a nicotinic acetylcholine receptor (nAChR) subtype and has real potential to positively impact human health. Major depressive disorder (MDD) is one of the most common psychiatric conditions and a main contributor to the overall global disease burden, with ˜16% of Alaska's population suffering from this condition. nAChRs are pentameric ligand gated ion channels which are abundantly expressed in the central nervous system (CNS) and modulate brain cholinergic tone. Hypercholinergic tone, in part caused by α7-nAChR activation, is associated with depression, and pharmacologically desensitizing α7-nAChRs improves depressive symptoms in mice. A recent effective depression treatment strategy is to promote brain-derived neurotrophic factor (BDNF) production. By reducing expression of programmed cell death 4 (Pdcd4) protein by small interfering RNA (siRNA) cargo delivered by a CPP, BDNF expression is enhanced and reverses depressive-like behaviors in mice. siRNAs are an excellent way to reduced protein expression but are rapidly broken down in the body, and have limited success crossing the blood brain barrier unassisted, restricting their therapeutic potential. To protect siRNAs from degradation, they can be conjugated to CPPs, which can facilitate CNS siRNA delivery. However, CPPs generally interact broadly with cellular macromolecules and lipid bilayers. This results in uncontrolled cargo delivery, causing undesired side effects and ultimately failing to alleviate disease burden. Disclosed herein is an example of the generation of an α7-nAChR selective CPP that can reduce α7-nAChR hyperactivity and carry Pdcd4 siRNA cargo that can be a useful dual therapy approach for individuals with MDD. This effort is stymied by a critical barrier to progress: an α7-nAChR selective CPP. To address this key issue, a novel, highly-selective CPP has been designed that functionally inhibits α7-nAChRs, while minimally targeting nine other nAChR subtypes. The data consistently show that the lead CPP is not cytotoxic and can transport a fluorophore into neuronal-like cells by an α7-nAChR dependent mechanism. It can now be determined if the CPP can move siRNA cargo into neuronal-like cells and the mammalian brain.

Provided are methods to: 1) Determine if the lead CPP can deliver siRNA into cultured cells. The approach is to use the neuronal-like neuroblastoma Neuro2a and peripheral human embryonic kidney (HEK 293) cell lines to determine if the lead CPP can deliver siRNA cargo into cells (live cell confocal microscopy). The siRNA is anticipated to decrease target-protein Pdcd4 abundance and cause increased BDNF expression (Western Blots and ELISA). 2) Define capability of lead CPP to deliver siRNA cargo into mouse CNS. Live animal image lead CPP complexed with siRNA tagged with the fluorophore fluorescein, and monitor CNS entrance and lifespan using fluorescent microscopy can be performed. Using harvested brain and key organ tissues, changes in Pdcd4 and BDNF expression can be determined by Western Blots and ELISA assays. Fixed brain tissue will be imaged using confocal microscopy to determine preferential locations of the complexed lead CPP/siPdcd4.

The cargo delivery potential of the lead CPP, which is disclosed throughout, can be determined.

### 1. Significance

MDD affects ˜280 million people globally, and has been ranked the third cause of disease burden by the World Health Organization. Alaska's burden is even proportionally greater, with ˜16% of Alaska's population suffering from depression. Current pharmacological treatment strategies are unsatisfactory in that at least 33-55% of people suffering from MDD are non-responsive to current antidepressant therapies. Depression is a complex disorder associated with alterations in neurotransmitter systems including hypercholinergic tone, and decreased BDNF expression. Common therapies are serotonin and norepinephrine reuptake inhibitors, which also antagonize nAChRs at clinically effective doses. nAChRs are pentameric ligand gated ions channels that activate in response to the neurotransmitter acetylcholine (ACh). The distribution of nAChRs is vast in the peripheral and central nervous system (CNS), where nAChR activation causes an influx of cations into the cell to affect membrane polarity and intracellular messenger cascades. Within the CNS, nAChRs are distributed throughout brain regions associated with depression including the ventral tegmental area (VTA), locus coeruleus, and dorsal raphe nucleus. α7-nAChRs are the second most abundantly expressed subtype in the CNS and regulate the depression-associated cholinergic ascending anti-inflammatory pathway.

nAChRs are therapeutic targets for MDD, chronic pain, and nicotine addiction. Ligands and peptides, which antagonize α7-nAChRs, have proven to be useful in MDD. For example, nicotine, a classical desensitizing nAChR ligand, has antidepressant properties in both preclinical and clinical studies. Additionally, methods to increase BDNF also result in depression symptom relief, including inhibition of Pdcd4 protein (an endogenous protein translation inhibitor). The connection between enhancement in BDNF expression and elevated mood is not surprising. Standard conventional antidepressant drugs and selective serotonin reuptake inhibitors (SSRIs) enhance BDNF mRNA levels and downstream signaling pathways. Because multiple brain systems are involved in MDD, dual therapy is likely to be advantageous, especially for those who have developed drug tolerance to traditional approaches, or who experience drug resistant depression. One possible avenue is to reduce the hypercholinergic tone while simultaneously upregulating BDNF expression. It is hypothesized that the disclosed CPP, a selective α7-nAChR antagonist, coupled with Pdcd4 siRNA can achieve this critical goal to help those suffering from MDD.

Using the disclosed peptides as a possible therapy is advantageous for several reasons. The use of the disclosed therapeutic peptides has increased yearly due to augmented specificity that is achievable with peptides versus small molecules. Additionally, CPPs can be coupled with cargo such as siRNAs allowing for CNS delivery. The lead CPP is ideal for the possible treatment of MDD as it shows high nAChR subtype specificity, is non-neurotoxic, and is able to enter into neuronal-like cells within 3 hrs post-exposure and is visually present up to 72 hrs. The lead CPP has promise to remedy depression treatment gaps, but needs to be further developed by determining if it can transport cargo, such as siRNA, into neuronal-like cells and into the mammalian brain, which are the objectives of this proposal.

### 2. Scientific Premise

Safe and successful delivery of therapeutics via CPPs, generated using a region of the neurotropic rabies virus glycoprotein (RVG), into the mouse CNS has been accomplished by a putative nAChR-mediated mechanism. RVG CPPs deliver cargo into mouse brain regions (e.g. cortex, striatum, thalamus), which express α4β2, α7, α6β2β3, and other nAChR subtypes. These studies provide evidence that RVG peptides are not nAChR subtype selective, a conclusion supported by the data (FIG. 1), and thus cargo delivery is not targeted. However, the RVG peptide does show a slight preference for the α7-nAChR, a property that has been enhanced with the lead CPP (FIG. 3).

RVG contains a neurotoxin-like region with significant sequence homology to snake α-neurotoxins, potent nAChR subtype selective antagonists. Snake toxins are proteins capable of discerning between nAChR subtypes, but are cytotoxic, lack stability, and are not readily bioavailable. Initially to generate our lead CPP, we combined regions of α-neurotoxins that facilitate subtype specificity with regions of the RVG peptide (FIG. 3A). Only the lead CPP met our positive hit criteria showing >80% inhibition of ACh-mediated currents at 100 μM peptide on no more than two subtypes, and <25% inhibition on other nAChRs (FIG. 3A). The parent RVG peptide prefers the α7-nAChR (>80% inhibition), but shows >25% inhibition on >five other subtypes (FIGS. 1 and 3A). Additionally, the lead CPP has low μM potency for α7-nAChRs (FIG. 3B).

Certain CPPs are cytotoxic, which is useful to kill cancerous cells. Previously tested RVG CPPs were not cytotoxic at the used concentrations, however, cytotoxicity was not specifically evaluated. the RVG and lead CPP were directly evaluated for cytotoxic effects using neuronal-like N2a and HEK cell lines, and have found parent RVG and lead CPP are not neuro- or cyto-toxic 24h post-incubation using α7-nAChR transfected and non-transfected cells (FIG. 5). Incubation with 15% DMSO kills cells, demonstrating changes in viability can be detected (FIG. 5C and D).

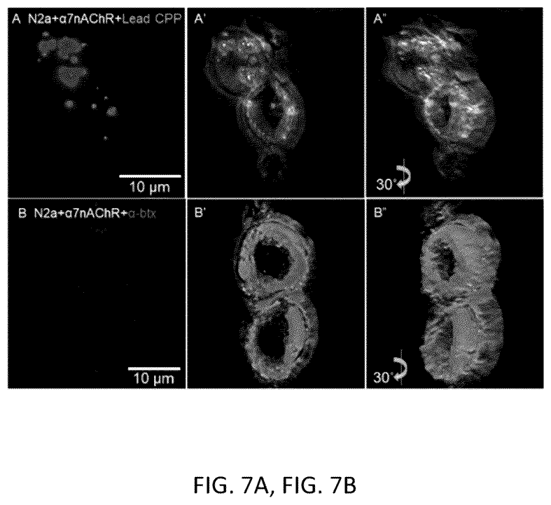

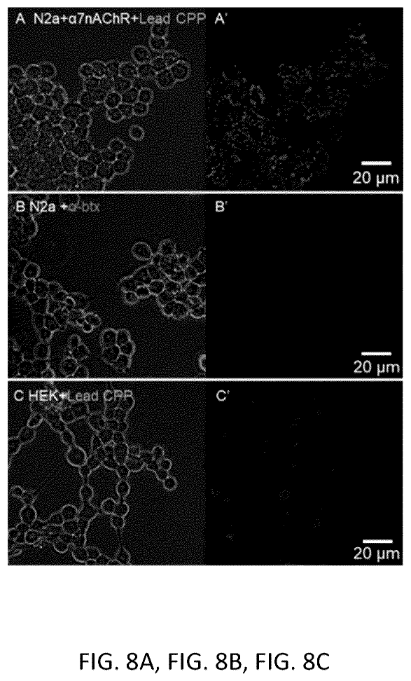

The lead CPP tagged with the fluorophore FITC (lead CPP-FITC) can efficiently enter into neuronal-like cells transfected with α7-nAChR subunit DNA, demonstrating successful transport of attached cargo into cells (FIG. 7A-A″). This key preliminary data provides supportive evidence that the lead CPP may be able to transport other materials, such as siRNAs, into cells. α-bungarotoxin (a-btx), an α7-nAChR selective neurotoxin is unable to enter into α7-nAChR transfected neuronal-like cells (FIG. 7B-B″), demonstrating the uniqueness of the lead CPP. Importantly, this mechanism is dependent on α7-nAChRs, as N2a cells transfected with α7-nAChR DNA are labeled abundantly with the lead CPP-FITC (FIG. 8A). As N2a's endogenously express α7-nAChRs (FIG. 8B), we verified the α7-nAChR requirements for cell entrance by demonstrating that the lead CPP-FITC is unable to target un-transfected HEK cells, which do not endogenously express α7-nAChRs (FIG. 8C)

CPPs derived from RVG have been used successfully to transport siRNA, nanoparticles, and other materials into mouse CNS. In a foundational paper, Kumar et al. (2007) initially showed that an RVG CPP could deliver antiviral siRNA and provided robust protection against fatal viral encephalitis in mice, without causing an immune or peptide antibody response. Live animal fluorescent microscopy assays have verified that RVG CPPs can enter the CNS and provide a lifespan landscape of movement and clearance of the CPP in the CNS and peripheral organs. At a 2 mg/kg dosage, a Cy5-tagged siPdcd4 complexed with an RVG CPP entered the mouse CNS and resided for at least 48 hrs. The delivered siPdcd4 was functional and decreased Pdcd4 mRNA expression 2-fold compared to negative control. Reduction in Pdcd4 protein caused an enhancement in BDNF, resulting in depressive symptom relief in a chronic restraint stress mouse model. These important studies demonstrate a proof of concept that RVG CPPs can effectively deliver potential therapeutic cargo into mouse CNS to induce positive physiological change. The lead CPP disclosed herein is highly selective for the α7-nAChR and more potent than previously used RVG CPPs, therefore providing a potential solution to this barrier, if the CPP can enter into the CNS.

Alternatives to current therapies for MDD have gained revitalized attention as evident by National Institutes of Health (NIH) and Department of Defense (DOD) funding calls in attempts to improve health burden and combat suicide. Upon successful completion of the project a detailed understanding of the siRNA delivery potential of the lead CPP in neuronal-like cells (Aim 1) and the mammalian brain (Aim 2) will be known. This significant contribution can have broad translational importance for the use of the novel CPP to deliver therapeutic siRNA cargo in an α7-nAChR subtype selective manner, reducing potential side effects, and providing the groundwork for a much-needed new MDD treatment strategy.

### 3. Innovation

The proposed research is innovative because it represents a substantial departure from existing non-target selective CPP technologies, by developing the lead α7-nAChR selective CPP. The lead CPP has the potential to solve CNS neuronal delivery of siRNAs. The status quo to using RVG CPPs to deliver CNS therapeutics assumes that RVG CPPs target α-btx-sensitive nAChRs. While the research and therapeutic potential are convincing as RVG peptides cross the blood brain barrier to deliver cargo into the mouse brain, these studies and the preliminary data demonstrate RVG CPPs are not nAChR subtype selective due to this peptide's penetrating affects in a variety of neuronal cell lines and brain regions. Devising fusion peptides that utilize the existing high affinity α-btx as a scaffold, as done with the lead CPP in this proposal, is a new avenue for increasing the utility of CPPs with macromolecule and thus cell-type specificity. Reducing off-target interactions of CPPs is likely to minimize potential side-effects and improve the safety profile for in vivo research and potential therapeutic applications.

Study outcomes can result in a new strategy to deliver therapeutic materials into the CNS. Ibis study proposes to deliver siRNA for Pdcd4 in efforts to determine if the novel CPP can: 1) deliver siRNA cargo into neuronal-like cells by an α7-nAChR mechanism, and 2) transport siRNA into the CNS and cause a cellular change by reducing expression of Pdcd4. Execution of these goals provides strong preliminary data for future applications to explore this strategy as a method to relive depressive symptoms in a mouse model. An additional application of the developed CPP is cargo delivery into the CNS in a non-invasive manner for research applications. Developing nAChR subtype selective CPPs could assist in targeted delivery of tagged liposomes, non-viral vectors, and adeno-associated viruses' (AAVs). Additionally, fluorescent tags can be added to the lead CPP to directly identify specific nAChRs facilitating surface expression identification (FIGS. 7 and 8). Finally, as multiple brain systems are involved in depression, the proposed dual therapy (lead CPP-9dR/siPdcd4) can be advantageous, especially for those who have developed drug tolerance to traditional approaches, or those experiencing drug resistant depression.

### 4. Approach

i. Aim 1. Determine if the Lead CPP can Deliver siRNA into Cultured Cells.

Provided are methods to determine if the lead CPP can deliver potential therapeutic cargo into neuronal-like cells. The approach is to visualize cell penetration of the lead CPP complexed with Pdcd4 siRNA tagged with 3′-Fluorescein (FL) (lead CPP-9dR/siPdcd4-FL) into a7-nAChR expressing N2a cells using live cell confocal microscopy. nAChR-null control HEK cells can also be exposed to the lead CPP/siRNA complex. To determine the siPdcd4 cellular effect, western blots and ELISA assays can be used to detect and quantify changes in Pcpd4 and BDNF expression. The rationale for this aim is that if the lead CPP can delivery siRNA into neuronal-like cells, similar outcomes may occur in mice.

a. Research Design.

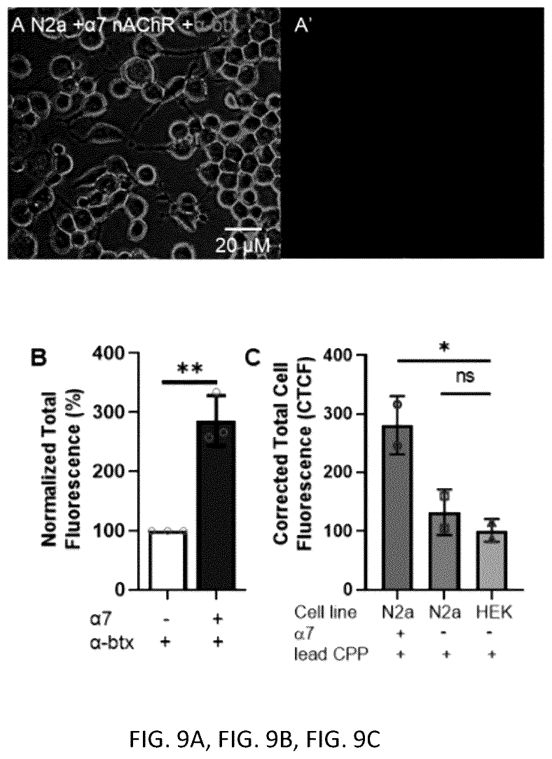

The lead CPP can be evaluated for its ability to deliver cargo material using neuronal-like N2a and peripheral-like HEK cell lines (American Type Culture Collection (ATCC), Manassas, VA). Peptide/siRNA complex formation: The nine D-Arg residues (9dR) will be added to the C-terminus of the lead CPP (YRKMWMPENPRLGTSCDAFCSSRGKVVELGRRRRRRRRR) during synthesis, as has been performed previously for other CPPs, to facilitate siPdcd4-FL (5′-GAGGCUAUGAGAGAAUUUATT-FL-3′) attachment (Horizon Discovery, Lafayette, LA) [25]. Note, FL will be used instead of FITC, as FITC is not available. The negative control peptide of similar length derived from the rabies viral matrix protein (RV-MAT-9dR, MNLLRKIVKNRRDEDTQKSSPASAPLDGGGGRRRRRRRRR) can be complexed with siPdcd4-FL, as this peptide has previously been determined to not be a CPP. As a positive control, lipofectamine will be used to transport un-complexed siPdcd4-FL into cells, as previously performed. As an additional negative control, cells can be exposed to siPdcd4-FL alone. Peptides can be purchased from ELIM Biopharmaceuticals, Inc. (Hayward, CA). Peptides and siRNAs used in Aim 1 and 2 can be sterile, in vivo quality, and stored lyophilized in the dark under −20° C. conditions. Solutions can be made fresh daily and solubilized in 2% DMSO and growing media. To complex siPdcd4-FL with lead CPP-9dR (lead CPP-9dR/siPdcd4-FITC), a molar ratio of 1:10 (siRNA:peptide) can be mixed in opti-MEM medium for 20 min at room temperature. siPdcd4 will be labelled with FL at the 3′-end of the sense strand to allow for visualization. Cell culture and siRNA transfection: To express α7-nAChRs, N2a and HEK cells will be transiently transfected using the Lipofectamine 2000 reagent (ThermoFisher Scientific) according to the manufacture's protocol (FIGS. 9A and B). α7-nAChRs express poorly, thus are co-transfected with chaperone protein NACHO in a 4:1 ratio. Cultured cells can be maintained in Eagle's Modified Eagle's Medium (EMEM) supplemented with 10% fetal bovine serum and 1X Penicillin-Streptomycin (VWR, Radnor, PA), at 37° C. and 5% CO₂ incubation conditions. 24h post-transfection, cells can be rinsed and 10 μM (IC50 value) of the complexed peptide/siRNA can be added. Images will be acquired at 3, 24, and 48 hrs post exposure. Tripled PBS washed live cells can be imaged (static and z-stacks) using a FLUOVIEW FV10i confocal laser scanning microscope (Olympus LS, Waltham, MA). Images can be deconvoluted and analyzed using ImageJ (NIH). To quantify changes in fluorescence, the mean integrated density of fluorescence of the cell groups was determined using 2D images. Fluorescent cells are free-hand traced and analyzed by measuring the region of interest area and integrated density, and subtracting out the background. The corrected total cell fluorescence (CTCF) of each cell is determined subtracting the integrated density from the area of the cell multiplied by the mean background fluorescence. Results are analyzed in GraphPad Prism 9.3 Software (San Diego, CA). An a priori power analysis was conducted (G*Power 3.1, Heinrich Heine University, Germany) for sample size estimation, based on preliminary data (N=2), which compared lead CPP-FITC CTCF values of α7-nAChR transfected vs. non-transfected cells (FIG. 10C). The preliminary data effect size was 4.7, with a significance criterion of a=0.05 and power=0.95. The minimum sample size needed with this effect size is N=3 (paired Student's t-test).

After 24 and 48 hrs post lead CPP-9dR/siPdcd4-FL exposure, changes in Pdcd4 and BDNF protein quantification can be measured and quantified using Western Blots and ELISA assays, respectively.

### (A)Western Blot Assay:

Cells will be washed with PBS and placed immediately on ice. Cells can be frozen in Radioimmunoprecipitation Assay (RIPA) lysis buffer containing cocktail proteinase inhibitor (VWR), and can be fully lysed using tissue ultrasonic grinder. Cellular lysates can be centrifuged at 13000 rpm for 20 min at 4° C., and supernatants can be collected. The bicinchoninic acid (BCA) protein assay kit (VWR) can be used to determine protein concentration. 30 pg/lane of protein from each replicate will be loaded on a 15% sodium dodecylsulfate-polyacrylamide gels (SDS-PAGE) and separated by electrophoresis. Proteins can be transferred to polyvinylidene difluoride (PVDF, MilliporeSigma, Burlington, MA) membranes using the semi-dry transfer method (15V, 1 hr). After blocking with 5% BSA for 1.5h at room temperature, the membrane can be incubated with primary antibodies for Pdcd4 (anti-Pdcd4, #9535S,) and β-actin (control, anti-β-actin, #4967) overnight at 4° C. (Cell Signaling Technology, Danvers, MA). The secondary horseradish peroxidase (HRP)-conjugated antibody (HRP-conjugated anti-mouse) can be incubated for 1.5h at room temperature with shaking (Jackson ImmunoResearch Laboratories, West Grove, PA). Bands can be visualized using an enhanced chemiluminescence (ECL) using the Amersham Storm 860 imager. The relative protein concentration can be analyzed using ImageJ software (NIH) and normalized to the β-actin control group.

### (B) ELISA:

For BDNF detection in cell supernatants, cellular fragments can be removed by centrifugation at 13000 rpm for 20 min at 4° C. and quantified by BDNF-specific Enzyme-Linked Immunosorbent Assay (ELISA) (Biolegend, San Diego, CA) according to manufacturer's instructions. Final BDNF concentration can be measured using optical density (OD) values using the Tecan Spark (Tecan Trading AG, Switzerland). An a priori power analysis (G*Power 3.1) was performed, based on preliminary data (N=3) from, which compared BDNF levels in control vs. siPdcd4 exposed BV2 cells (effect size=3.3, a=0.05, and power=0.95). Minimum sample size needed is N=4 (paired Student's t-test). Data can be analyzed using Student's t-test to compare pairs of groups or by one-way ANOVA with Tukey's multiple comparison tests to evaluate means of three or more groups using GraphPad Prism.

b. Expected Results

The lead CPP-9dR can facilitate delivery of siPdcd4-FL into α7-nAChR transfected cells, but not into non-transfected HEK cells, which are nAChR null. siPdcd4 can knock down expression of Pdcd4 protein and enhance BDNF production.

I. Aim 2. Define Capability of Lead CPP to Deliver siRNA Cargo into Mouse CNS.

The objective is to evaluate the CNS entry and siRNA delivery potential of the lead CPP using mice. The working hypothesis that siPdcd4 can be transported into the mouse CNS to reduce Pdcd4 mRNA abundance, which will in turn enhance BDNF expression, is tested. The approach is to visualize CNS penetration of the lead CPP complexed with siPdcd4-FL at several time points using in vivo microscopy as previously performed for RVG CPPs. Harvested brain and key organ tissues can be imaged ex vivo, and changes in Pdcd4 and BDNF expression can be determined by Western Blots and ELISA assays. Fixed brains can be imaged using confocal microscopy to determine preferential locations of the complexed lead CPP-9dR/siPdcd4-FL. The rationale for this aim is that successful mammalian CNS entrance and siRNA delivery can provide a proof of concept that a CPP has been developed with high potential for therapeutic and research applications. Upon completion, it can be determined that the CNS entrance and cargo delivery potential of the lead CPP, making significant progress towards the development of a new therapy to aid in the treatment of major depressive disorder.

a. Research Design.

In vivo mouse injection of lead CPP-d9R/siPdcd4-FL complex: Sterile 50 pg (2 mg/kg) of lead CPP-d9R/siPdcd4-FL or vehicle 5% glucose (negative control) can be injected into mice though the tail vein as previously accomplished. Because detecting changes in fluorescence, and pigmented skin of C57BL16 mice has been shown to contribute to fluorescent responses, B6(Cg)-Tyrc-2J/J male and female mice (aged 4-6 weeks) can be used as these are C57BU16J albino mice that carry a mutation in the tyrosinase gene (The Jackson Laboratory, Sacramento, CA). At the time of injection, animals will be anesthetized with 2% isoflurane, and the hair on the head and back can be removed to facilitate fluorescence detection. Six hours post-injection, animals can be imaged using a Leica M205 FA with a Leica X-Cite 120Q illuminator imaging system with an in-house built dark chamber to track the movement of the lead CPP-9dR/siPdcd4-FL through the mice. After each imaging session, animals can be returned to their home cages and monitored until fully awake and active. Imaging sessions can be repeated at 24, 48, and 72 hr. These initial imaging sessions allow for determining the optimal time point for lead CPP-9dR/siPdcd4-FL complex delivery into the CNS. Based on previous work studying RVG CPPs, 24 or 48 hr post-injection is when the highest fluorescence detection occurs. After the last imaging session, animals will be euthanized by isoflurane overdose, followed by decapitation. Brain, lung, liver, kidneys, and heart can be collected for further analysis of siRNA delivery. The brain can be divided to separate the left and right hemispheres. The left hemisphere can be fixed in 4% paraformaldehyde and analyzed for lead CPP-d9R/siPdcd4-FL in brain regions including cortex, VTA, hippocampus, striatum, and thalamus using a FLUOVIEW FV10i confocal laser scanning microscope. The right hemisphere and other tissues can be washed with PBS and placed on ice until frozen quickly in RIPA lysis buffer with protease cocktail, as described immediately above for cultured cells. Once the peak fluorescence detection time has been determined from the first set of 8 animals, the above imaging sessions can be performed again, but with tissue harvesting occurring at that time point. To determine changes in Pdcd4 or BDNF expression, western blots and ELISA assays can be performed as described above in Aim 1. For each of the three treatments, N=8 with 4 males and 4 females can be used for a total of 24 animals. Power analysis and blinding: An a priori power analysis was conducted (G*Power 3.1) for sample size estimation, based on preliminary data (N=3) from, which compared BDNF/0-actin levels in control vs. 50 pg of RVG/siPdcd4-Cy5 exposed mice (effect size=3.2, a=0.05, and power=0.95). The minimum sample size needed with this effect size is N=4 (paired Student's t-test). Thus, a sample size of N=4 per sex is adequate. Prior to performing brain imaging analysis, samples can be re-labeled to ensure the student is blinded.

b. Results

The lead CPP-9dR can facilitate siPdcd4-FL delivery into mouse CNS, and can accumulate in the hippocampus and prefrontal cortex as these brain regions highly express 07-nAChRs. Conversely, the thalamus minimally expresses α7-nAChRs and thus low FL detection is expected. The lead CPP-9dR/siRdcd4-FL can reach maximal levels in the brain at 24 or 48 hr post-injection. Tissue analyses are anticipated to reveal the siPdcd4 decreases Pdcd4 expression and enhance BDNF production.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Ayoo, K., et al., The opioid crisis in North America: facts and future lessons for Europe. Anaesthesiol Intensive Ther, 2020. 52(2): p. 139-147.
2. Rabenstein, R. L., B. J. Caldarone, and M. R. Picciotto, The nicotinic antagonist mecamylamine has antidepressant-like effects in wild-type but not beta2-or alpha7-nicotinic acetylcholine receptor subunit knockout mice. Psychopharmacology (Berl), 2006. 189(3): p. 395-401.
3. Shytle, R. D., et al., Nicotinic acetylcholine receptors as targets for antidepressants. Mol Psychiatry, 2002. 7(6): p. 525-35.
4. Satkunanathan, N., et al., Alpha-conotoxin Vcl.1 alleviates neuropathic pain and acceleratesfunctional recovery ofinjured neurones. Brain Res, 2005. 1059(2): p. 149-58.
5. Pacini, A., et al., The alpha9alpha10 nicotinic receptor antagonist alpha-conotoxin RgIA prevents neuropathic pain induced by oxaliplatin treatment. Exp Neurol, 2016. 282: p. 37-48.
6. Christensen, S. B., et al., RgIA4 Potently Blocks Mouse alpha9alpha10 nAChRs and Provides Long Lasting Protection against Oxaliplatin-Induced Cold Allodynia. Front Cell Neurosci, 2017. 11: p. 219.
7. AiSharari, S. D., et al., The alpha9alpha10 nicotinic acetylcholine receptors antagonist alpha-conotoxin RgIA reverses colitis signs in murine dextran sodium sulfate model. Eur J Pharmacol, 2020. 883: p. 173320.
8. Giribaldi, J., et al., Backbone Cyclization Turns a Venom Peptide into a Stable and Equipotent Ligand at Both Muscle and Neuronal Nicotinic Receptors. J Med Chem, 2020. 63(21): p. 12682-12692.
9. Kumar, P., et al., Transvascular delivery of small interfering RNA to the central nervous system. Nature, 2007. 448(7149): p. 39-43.
10. Liu, Y., et al., Brain-targeting gene delivery and cellular internalization mechanisms for modified rabies virus glycoprotein RVG29 nanoparticles. Biomaterials, 2009. 30(25): p. 4195-202.
11. Liu, Y., et al., Targeted exosome-mediated delivery of opioid receptor Mu siRNA for the treatment of morphine relapse. Sci Rep, 2015. 5: p. 17543.
12. Lau, J. L. and M. K. Dunn, Therapeutic peptides: Historical perspectives, current development trends, and future directions. Bioorg Med Chem, 2018. 26(10): p. 2700-2707.
13. Taly, A., et al., Nicotinic receptors: allosteric transitions and therapeutic targets in the nervous system. Nat Rev Drug Discov, 2009. 8(9): p. 733-50.
14. Hogg, R. C. and D. Bertrand, Nicotinic acetylcholine receptors as drug targets. Curr Drug Targets CNS Neurol Disord, 2004. 3(2): p. 123-30.
15. Gotti, C., M. Zoli, and F. Clementi, Brain nicotinic acetylcholine receptors: native subtypes and their relevance. Trends Pharmacol Sci, 2006. 27(9): p. 482-91.
16. Duarte, Y., et al., Different Classes of Antidepressants Inhibit the Rat alphα7 Nicotinic Acetylcholine Receptor by Interacting within the Ion Channel: A Functional and Structural Study. Molecules, 2021. 26(4).
17. Luo, S., et al., Cloning, synthesis, and characterization of alphaO-conotoxin GeXIVA, a potent alpha9alphal0 nicotinic acetylcholine receptor antagonist. Proc Natl Acad Sci USA, 2015. 112(30): p. E4026-35.
18. Dutertre, S., A. Nicke, and V. I. Tsetlin, Nicotinic acetylcholine receptor inhibitors derived from snake and snail venoms. Neuropharmacology, 2017. 127: p. 196-223.
19. Barber, C. M., G. K. Isbister, and W. C. Hodgson, Alpha neurotoxins, Toxicon, 2013. 66: p. 47-58.
20. Kasheverov, V. T. a. I., Peptide and Protein Neurotoxin Toolbox in Research on Nicotinic Acetylcholine Receptors, in Neurochemistry, T. Heinbockel, Editor. 2014, InTech.
21. Andreasen, J. T., et al., Antidepressant-like effects of nicotinic acetylcholine receptor antagonists, but not agonists, in the mouse forced swim and mouse tail suspension tests. J Psychopharmacol, 2009. 23(7): p. 797-804.
22. Mineur, Y. S., et al., Hippocampal alphα7 nicotinic ACh receptors contribute to modulation of depression-like behaviour in C57BL/6J mice. Br J Pharmacol, 2018. 175(11): p. 1903-1914.
23. Semenkovich, K., et al., Prescription Opioid Analgesics Increase Risk of Major Depression: New Evidence, Plausible Neurobiological Mechanisms and Management to Achieve Depression Prophylaxis. Mo Med, 2014. 111(2): p. 148-154.
24. Huang, S., et al., Complex between alpha-bungarotoxin and an alphα7 nicotinic receptor ligand-binding domain chimaera. Biochem J, 2013. 454(2): p. 303-310.
25. Bourne, Y., et al., Crystal structure of a Cbtx-AChBP complex reveals essential interactions between snake alphα-neurotoxins and nicotinic receptors. EMBO J, 2005. 24(8): p. 1512-22.
26. Fruchart-Gaillard, C., et al., Experimentally based model of a complex between a snake toxin and the alpha 7nicotinic receptor. Proc Natl Acad Sci USA, 2002. 99(5): p. 3216-21.
27. Chiappinelli, V. A., et al., Binding of native kappα-neurotoxins and site-directed mutants to nicotinic acetylcholine receptors. Toxicon, 1996. 34(11-12): p. 1243-56.
28. Yang, Y. J., et al., Production and characterization of a fusion peptide derived from the rabies virus glycoprotein (RVG29). Protein Expr Purif, 2014. 104: p. 7-13.
29. Reitstetter, R., RJ. Lukas, and R Gruener, Dependence ofnicotinic acetylcholine receptor recoveryfrom desensitization on the duration of agonist exposure. J Pharmacol Exp Ther, 1999. 289(2): p. 656-60.
30. Papke, R. L., et al., Activation and desensitization of nicotinic alpha7-type acetylcholine receptors by benzylidene anabaseines and nicotine. J Pharmacol Exp Ther, 2009. 329(2): p. 791-807.
31. Arias, H. R., et al., Tricyclic antidepressants inhibit hippocampal alpha7* and alpha9alpha10 nicotinic acetylcholine receptors by different mechanisms. Int J Biochem Cell Biol, 2018. 100: p. 1-10.
32. Gotti, C., et al., Structural and functional diversity of native brain neuronal nicotinic receptors. Biochem Pharmacol, 2009. 78(7): p. 703-11.
33. Elgoyhen, A. B., et al., Alpha 9: an acetylcholine receptor with novel pharmacological properties expressed in rat cochlear hair cells. Cell, 1994. 79(4): p. 705-15.
34. Morley, B. J., P. Whiteaker, and A. B. Elgoyhen, Commentary: Nicotinic Acetylcholine Receptor alpha9 and alpha10 Subunits Are Expressed in the Brain of Mice. Front Cell Neurosci, 2018. 12: p. 104.
35. Lips, K. S., U. Pfeil, and W. Kummer, Coexpression of alpha 9 and alpha 10 nicotinic acetylcholine receptors in rat dorsal root ganglion neurons. Neuroscience, 2002. 115(1): p. 1-5.
36. Callaghan, B. and D. J. Adams, Analgesic alpha-conotoxins Vcl.I and RgIA inhibit N-type calcium channels in sensory neurons of alpha9 nicotinic receptor knockout mice. Channels (Austin), 2010. 4(1): p. 51-4.
37. Rosas-Ballina, M. and K. J. Tracey, Cholinergic control of inflammation. J Intern Med, 2009. 265(6): p. 663-79.
38. Fujii, T., et al., Expression and Function of the Cholinergic System in Immune Cells. Front Immunol, 2017. 8: p. 1085.
39. Liu, Q., et al., Distinctive Roles for alpha7*-and alpha9*-Nicotinic Acetylcholine Receptors in Inflammatory and Autoimmune Responses in the Murine Experimental Autoimmune Encephalomyelitis Model of Multiple Sclerosis. Front Cell Neurosci, 2017. 11: p. 287.
40. Hone, A. J. and J. M. McIntosh, Nicotinic acetylcholine receptors in neuropathic and inflammatory pain. FEBS Lett, 2018. 592(7): p. 1045-1062.
41. Emery, P. C., K. G. Wilson, and J. Kowal, Major depressive disorder and sleep disturbance in patients with chronic pain. Pain Res Manag, 2014. 19(1): p. 35-41.
42. Huey, R, et al., Targeted drug delivery system to neural cells utilizes the nicotinic acetylcholine receptor. Int J Pharm, 2017. 525(1): p. 12-20.
43. Alvarez-Erviti, L., et al., Delivery of siRNA to the mouse brain by systemic injection of targeted exosomes. Nat Biotechnol, 2011. 29(4): p. 341-5.
44. Gotti, C., et al., Expression of nigrostriatal alpha 6-containing nicotinic acetylcholine receptors is selectively reduced, but not eliminated, by beta 3 subunit gene deletion. Mol Pharmacol, 2005. 67(6): p. 2007-15.
45. Hogg, RC., M. Raggenbass, and D. Bertrand, Nicotinic acetylcholine receptors: from structure to brain function. Rev Physiol Biochem Pharmacol, 2003. 147: p. 1-46.
46. Quik, M., X. A. Perez, and S. R Grady, Role of alpha6 nicotinic receptors in CNS dopaminergic function: relevance to addiction and neurological disorders. Biochem Pharmacol, 2011. 82(8): p. 873-82.
47. Grady, S. R., et al., The subtypes of nicotinic acetylcholine receptors on dopaminergic terminals of mouse striatum. Biochem Pharmacol, 2007. 74(8): p. 1235-46.
48. Gotti, C., et al., Nicotinic acetylcholine receptors in the mesolimbic pathway: primary role of ventral tegmental area alpha6beta2* receptors in mediating systemic nicotine effects on dopamine release, locomotion, and reinforcement. J Neurosci, 2010. 30(15): p. 5311-25.
49. Luetje, C. W., Getting past the asterisk: the subunit composition of presynaptic nicotinic receptors that modulate striatal dopamine release. Mol Pharmacol, 2004. 65(6): p. 1333-5.
50. Lentz, T. L., Structure-function relationships of curaremimetic neurotoxin loop 2 and of a structurally similar segment of rabies virus glycoprotein in their interaction with the nicotinic acetylcholine receptor. Biochemistry, 1991. 30(45): p. 10949-57.
51. Antil-Delbeke, S., et al., Molecular determinants by which a long chain toxin from snake venom interacts with the neuronal alpha 7-nicotinic acetylcholine receptor. J Biol Chem, 2000. 275(38): p. 29594-601.
52. Noviello, C. M., et al., Structure and gating mechanism of the alphα7 nicotinic acetylcholine receptor. Cell, 2021. 184(8): p. 2121-2134 e13.
53. Rustici, M., et al., A model of the rabies virus glycoprotein active site. Biopolymers, 1993. 33(6): p. 961-9.
54. Faber, M., et al., A single amino acid change in rabies virus glycoprotein increases virus spread and enhances virus pathogenicity. J Virol, 2005. 79(22): p. 14141-8.
55. Neundorf, I., et al., Fusion of a Short HA2-Derived Peptide Sequence to Cell-Penetrating Peptides Improves Cytosolic Uptake, but Enhances Cytotoxic Activity. Pharmaceuticals (Basel), 2009. 2(2): p. 49-65.
56. Kilk, K., et al., Analysis of in vitro toxicity of five cell-penetrating peptides by metabolic profiling. Toxicology, 2009. 265(3): p. 87-95.
57. Prasampun, S., et al., EAvenoming bites by kraits: the biological basis of treatment-resistant neuromuscular paralysis. Brain, 2005. 128(Pt 12): p. 2987-96.
58. Yan, Z., et al., Depression and Opioid Misuse in Elderly Individuals With Chronic Pain: A Latent Class Analysis. Pain Manag Nurs, 2022.
59. Rupprecht, C. E., C A. Hanlon, and T. Hemachudha, Rabies re-examined. Lancet Infect Dis, 2002. 2(6): p. 327-43.
60. Salminen, O., et al., Subunit composition and pharmacology of two classes of striatal presynaptic nicotinic acetylcholine receptors mediating dopamine release in mice. Mol Pharmacol, 2004. 65(6): p. 1526-35.
61. Champtiaux, N., et al., Subunit composition of functional nicotinic receptors in dopaminergic neurons investigated with knock-out mice. J Neurosci, 2003. 23(21): p. 7820-9.
62. Philip, N. S., et al., Nicotinic acetylcholine receptors and depression: a review of the preclinical and clinical literature. Psychopharmacology (Berl), 2010. 212(1): p. 1-12.
63. Ross, S. A., et al., Phenotypic characterization of an alpha 4 neuronal nicotinic acetylcholine receptor subunit knock-out mouse. J Neurosci, 2000. 20(17): p. 6431-41.
64. Picciotto, M. R., et al., Mood and anxiety regulation by nicotinic acetylcholine receptors: A potential pathway to modulate aggression and related behavioral states. Neuropharmacology, 2015. 96(Pt B): p. 235-43.
65. King, S. L., B. J. Caldarone, and M. R. Picciotto, Beta2-subunit-containing nicotinic acetylcholine receptors are critical for dopamine-dependent locomotor activation following repeated nicotine administration. Neuropharmacology, 2004. 47 Suppl 1: p. 132-9.
66. Weltzin, M. M., et al., Distinctive effects of nicotinic receptor intracellular-loop mutations associated with nocturnal frontal lobe epilepsy. Neuropharmacology, 2016. 102: p. 158-73.
67. Weltzin, M. M. and M. K. Schulte, Desformylflustrabromine Modulates alpha4beta2 Neuronal Nicotinic Acetylcholine Receptor High-and Low-Sensitivity Isoforms at Allosteric Clefts Containing the beta2 Subunit. J Pharmacol Exp Ther, 2015. 354(2): p. 184-94.
68. Nelson, M. E., et al., Alternate stoichiometries of alpha4beta2 nicotinic acetylcholine receptors. Mol Pharmacol, 2003. 63(2): p. 332-41.
69. Moretti, M., et al., The novel alpha7beta2-nicotinic acetylcholine receptor subtype is expressed in mouse and human basal forebrain: biochemical and pharmacological characterization. Mol Pharmacol, 2014. 86(3): p. 306-17.
70. Papke, R. L., L. P. Dwoskin, and P. A. Crooks, The pharmacological activity of nicotine and nornicotine on nAChRs subtypes: relevance to nicotine dependence and drug discovery. J Neurochem, 2007. 101(1): p. 160-7.
71. George, A. A., et al., Isoform-specificmechanismsofalpha3beta4*-nicotinicacetylcholine receptor modulation by the prototoxin lynx1. FASEB J, 2017. 31(4): p. 1398-1420.
72. Lucero, L. M., et al., Differential alpha4(+)/(−)beta2Agonist-binding Site Contributions to alpha4beta2 Nicotinic Acetylcholine Receptor Function within and between Isoforms. J Biol Chem, 2016. 291(5): p. 2444-59.
73. Kim, J. S., et al., Synthesis of desformylflustrabromine and its evaluation as an alpha4beta2 and alphα7 nACh receptor modulator. Bioorg Med Chem Lett, 2007. 17(17): p. 4855-60.
74. Luo, S., et al., A novel alpha4/7-conotoxin LvIA from Conus lividus that selectively blocks alpha3beta2 vs. alpha6/alpha3beta2beta3 nicotinic acetylcholine receptors. FASEB J, 2014. 28(4): p. 1842-53.
75. Weltzin, M. M., Y. Huang, and M. K. Schulte, Allosteric modulation of alpha4beta2 nicotinic acetylcholine receptors by HEPES. Eur J Pharmacol, 2014. 732: p. 159-68.
76. Weltzin, M. M. and M. K. Schulte, Pharmacological characterization of the allosteric modulator desformylflustrabromine and its interaction with alpha4beta2 neuronal nicotinic acetylcholine receptor orthosteric ligands. J Pharmacol Exp Ther, 2010. 334(3): p. 917-26.
77. Thoulouze, M. I., et al., The neural cell adhesion molecule is a receptor for rabies virus. J Virol, 1998. 72(9): p. 7181-90.
78. Wang, J., et al., Metabotropic glutamate receptor subtype 2 is a cellular receptor for rabies virus. PLoS Pathog, 2018. 14(7): p. e1007189.
79. Shuai, L., et al., Integrin beta1 Promotes Peripheral Entry by Rabies Virus. J Virol, 2020. 94(2).
80. Nichols, W. A., et al., Mutation Linked to Autosomal Dominant Nocturnal Frontal Lobe Epilepsy Reduces Low-Sensitivity alpha4beta2. and Increases alpha5alpha4beta2. Nicotinic Receptor Surface Expression. PLoS One, 2016. 11(6): p. e0158032.
81. Kweon, H. J., et al., NACHO Engages N-Glycosylation ER Chaperone Pathways for alphα7 Nicotinic Receptor Assembly. Cell Rep, 2020. 32(6): p. 108025.
82. Millar, N. S., RIC-3: a nicotinic acetylcholine receptor chaperone. Br J Pharmacol, 2008. 153 Suppi 1: p. S177-83.
83. Williams, M. E., et al., Ric-3 promotes functional expression of the nicotinic acetylcholine receptor alpha7subunit in mammalian cells. J Biol Chem, 2005. 280(2): p. 1257-63.
1. Organization, W. H. Depression. [11/19/2022]; Available from: https://www.who.int/news-room/fact-sheets/detail/depression.
2. Malhi, G. S. and J. J. Mann, Depression. Lancet, 2018. 392(10161): p. 2299-2312.
3. Foundation, U. H. America's Health Rankings Depression Annual Report 11/19/2022]; Available from: https-/www americashealthrankings.org/explore/annual/measure/Depression_a/state/A K.
4. Jick, H., J. A. Kaye, and S. S. Jick, Antidepressants and the risk of suicidal behaviors. JAMA, 2004. 292(3): p. 338-43.
5. Wiles, N., et al., Clinical effectiveness and cost-effectiveness of cognitive behavioural therapy as an adjunct to pharmacotherapy for treatment-resistant depression in primary care: the CoBalT randomised controlled trial. Health Technol Assess, 2014. 18(31): p. 1-167, vii-viii.
6. Shytle, R. D., et al., Nicotinic acetylcholine receptors as targets for antidepressants. Mol Psychiatry, 2002. 7(6): p. 525-35.
7. Taly, A., et al., Nicotinic receptors: allosteric transitions and therapeutic targets in the nervous system. Nat Rev Drug Discov, 2009. 8(9): p. 733-50.
8. Philip, N. S., et al., The nicotinic acetylcholine receptor as a target for antidepressant drug development. ScientificWorldJoumal, 2012. 2012: p. 104105.
9. Miller, A. H., V. Maletic, and C. L. Raison, Inflammation and its discontents: the role of cytokines in the pathophysiology of major depression. Biol Psychiatry, 2009. 65(9): p. 732-41.
10. Shytle, R. D., et al., Cholinergic modulation of microglial activation by alpha α7 nicotinic receptors. J Neurochem, 2004. 89(2): p. 337-43.
11. Gallowitsch-Puerta, M. and V A. Pavlov, Neuro-immune interactions via the cholinergic anti-inflammatory pathway. Life Sci, 2007. 80(24-25): p. 2325-9.
12. Pu, Y., et al., A role of the subdiaphragmatic vagus nerve in depression-like phenotypes in mice after fecal microbiota transplantation from Chrnα7 knock-out mice with depression-like phenotypes. Brain Behav Immun, 2021.94: p. 318-326.
13. Hogg, R. C. and D. Bertrand, Nicotinic acetylcholine receptors as drug targets. Curr Drug Targets CNS Neurol Disord, 2004. 3(2): p. 123-30.
14. Gotti, C., M. Zoli, and F. Clementi, Brain nicotinic acetylcholine receptors: native subtypes and their relevance. Trends Pharmacol Sci, 2006. 27(9): p. 482-91.
15. Rabenstein, R. L., B. J. Caldarone, and M. R. Picciotto, The nicotinic antagonist mecamylamine has antidepressant-like effects in wild-type but not beta2-or alpha7-nicotinic acetylcholine receptor subunit knockout mice. Psychopharmacology (Berl), 2006. 189(3): p. 395-401.
16. Duarte, Y., et al., Different Classes of Antidepressants Inhibit the Rat alphα7 Nicotinic Acetylcholine Receptor by Interacting within the Ion Channel: A Functional and Structural Study. Molecules, 2021. 26(4).
17. Luo, S., et al., Cloning, synthesis, and characterization of alphaO-conotoxin GeXIVA, a potent alpha9alpha10 nicotinic acetylcholine receptor antagonist. Proc Natl Acad Sci U S A, 2015. 112(30): p. E4026-35.
18. Andreasen, J. T., et al., Antidepressant-like effects of nicotinic acetylcholine receptor antagonists, but not agonists, in the mouse forced swim and mouse tail suspension tests. J Psychopharmacol, 2009. 23(7): p. 797-804.
19. Mineur, Y. S., et al., Hippocampal alphα7 nicotinic ACh receptors contribute to modulation of depression-like behaviour in C57BL6J mice. Br J Pharmacol, 2018. 175(11): p. 1903-1914.
20. Semba, J., et al., Antidepressantlike effects of chronic nicotine on learned helplessness paradigm in rats. Biol Psychiatry, 1998. 43(5): p. 389-91.
21. Tizabi, Y., et al., Antidepressant effects of nicotine in an animal model of depression. Psychopharmacology (Berl), 1999. 142(2): p. 193-9.
22. Salin-Pascual, R. J., Relationship between mood improvement and sleep changes with acute nicotine administration in non-smoking major depressed patients. Rev Invest Clin, 2002. 54(1): p. 36-40.
23. McClemon, F. J., et al., Transdermal nicotine attenuates depression symptoms in nonsmokers: a double-blind, placebo-controlled trial. Psychopharmacology (Ber), 2006. 189(1): p. 125-33.
24. Li, Y., et al., Programmed cell death 4 as an endogenous suppressor of BDNF translation is involvedin stress-induced depression. Mol Psychiatry, 2021. 26(6): p. 2316-2333.
25. Jia, Y., et al., The brain targeted delivery of programmed cell death 4 specific siRNA protects mice from CRS-induced depressive behavior. Cell Death Dis, 2021. 12(11): p. 1077.
26. Suzuki, C., et al., PDCD4 inhibits translation initiation by binding to eIF4A using both its MA3 domains. Proc Natl Acad Sci USA, 2008.105(9): p. 3274-9.
27. Nibuya, M., S. Morinobu, and R. S. Duman, Regulation of BDNF and trkB mRNA in rat brain by chronic electroconvulsive seizure and antidepressant drug treatments. J Neurosci, 1995. 15(11): p. 7539-47.
28. Duman, R. S. and L. M. Monteggia, A neurotrophic model for stress-related mood disorders. Biol Psychiatry, 2006. 59(12): p. 1116-27.
29. Schmidt, H. D. and R. S. Duman, Peripheral BDNF produces antidepressant-like effects in cellular and behavioralmodels. Neuropsychopharmacology, 2010. 35(12): p. 2378-91.
30. Lau, J. L. and M. K. Dunn, Therapeutic peptides: Historical perspectives, current development trends, and future directions. Bioorg Med Chem, 2018. 26(10): p. 2700-2707.
31. Liu, Y., et al., Targeted exosome-mediated delivery of opioid receptor Mu siRNA for the treatment of morphine relapse. Sci Rep, 2015.5: p. 17543.
32. Liu, Y., et al., Brain-targeting gene delivery and cellular internalization mechanisms for modified rabies virus glycoprotein RVG29 nanopartices. Biomaterials, 2009. 30(25): p. 4195-202.
33. Huey, R., et al., Targeted drug delivery system to neural cells utilizes the nicotinic acetylcholine receptor. Int J Pharm, 2017. 525(1): p. 12-20.
34. Kumar, P., et al., Transvascular delivery of small interfering RNA to the central nervous system. Nature, 2007. 448(7149): p. 39-43.
35. Alvarez-Erviti, L., et al., Delivery of siRNA to the mouse brain by systemic injection of targeted exosome. Nat Biotechnol, 2011. 29(4): p. 341-5.
36. Gotti, C., et al., Expression of nigrostriatal alpha 6-containing nicotinic acetylcholine receptors is selectively reduced, but not eliminated, by beta 3 subunit gene deletion. Mol Pharmacol, 2005. 67(6): p. 2007-15.
37. Hogg, R. C., M. Raggenbass, and D. Bertrand, Nicotinic acetylcholine receptors: from structure to brain function. Rev Physiol Biochem Pharmacol, 2003. 147: p. 1-46.
38. Quik, M., XA. Perez, and S. R. Grady, Role of alpha6 nicotinic receptors in CNS dopaminergic function: relevance to addiction and neurological disorders. Biochem Pharmacol, 2011. 82(8): p. 873-82.
39. Grady, S. R., et al., The subtypes of nicotinic acetylcholine receptors on dopaminergic terminals of mouse striatum. Biochem Pharmacol, 2007. 74(8): p. 1235-46.
40. Gotti, C., et al., Nicotinic acetylcholine receptors in the mesolimbic pathway: primary role of ventral tegmental area alpha6beta2* receptors in mediating systemic nicotine effects on dopamine release, locomotion, and reinforcement. J Neurosci, 2010. 30(15): p. 5311-25.
41. Luetje, C. W., Getting past the asterisk: the subunit composition of presynaptic nicotinic receptors that modulate striatal dopamine release. Mol Pharmacol, 2004. 65(6): p. 1333-5.
42. Barber, C. M., G. K. Isbister, and W. C. Hodgson, Alpha neurotoxins. Toxicon, 2013. 66: p. 47-58.
43. Kasheverov, V. T. a. I., Peptide and Protein Neurotoxin Toolbox in Research on Nicotinic Acetylcholine Receptors, in Neurochemistry, T. Heinbockel, Editor. 2014, InTech.
44. Dutertre, S., A. Nicke, and V. I. Tsetlin, Nicotinic acetylcholine receptor inhibitors derived from snake and snail venoms. Neuropharmacology, 2017. 127: p. 196-223.
45. Neundorf, I., et al., Fusion of a Short HA2-Derived Peptide Sequence to Cell-Penetrating Peptides Improves Cytosolic Uptake, but Enhances Cytotoxic Activity. Pharmaceuticals (Basel), 2009. 2(2): p. 49-65.
46. Kilk, K., et al., Analysis of in vitro toxicity of five cell-penetrating peptides by metabolic profiling. Toxicology, 2009. 265(3): p. 87-95.
47. Yang, Y. J., et al., Production and characterization of a fusion peptide derived from the rabies virus glycoprotein (RVG29). Protein Expr Purif, 2014. 104: p. 7-13.
48. Oppong-Damoah, A., et al., Nanoparticle encapsulation increases the brain penetrance and duration of action of intranasal oxytocin. Horm Behav, 2019. 108: p. 20-29.
49. You, L., et al., Targeted Brain Delivery of Rabies Virus Glycoprotein 29-Modified Deferoxamine-Loaded Nanoparticles Reverses Functional Deficits in Parkinsonian Mice. ACS Nano, 2018. 12(5): p. 4123-4139.
50. Kim, J. Y., et al., Brain-targeted delivery of protein using chitosan-and RVG pep6de-conjugated, pluronic-based nano-carrier. Biomaterials, 2013. 34(4): p. 1170-8.
51. Xu, J., et al., Cell-penetrating peptide: a means of breaking through the physiological barriers of different tissues and organs. J Control Release, 2019. 309: p. 106-124.
52. Xie, Y. L., et al., The use of melittin to enhance transgene expression mediated by recombinant adeno-associated virus serotype 2 vectors both in vitro and in vivo. J Integr Med, 2022.
53. Meng, Y., et al., Cell-penetrating peptides enhance the transduction of adeno-associated virus serotype 9 in the central nervous system. Mol Ther Methods Clin Dev, 2021. 21: p. 28-41.
54. Shalaby, K. E., et al., Development of non-viral vectors for neuronal-targeted delivery of CRISPR-Cas9 RNA-proteins as a therapeutic strategy for neurological disorders. Biomater Sci, 2022. 10(17): p. 4959-4977.
55. Gong, C., et al., Target delivery of a gene into the brain using the RVG29-oligoarginine peptide. Biomaterials, 2012. 33(12): p. 3456-63.
56. Kweon, H. J., et al., NACHO Engages N-Glycosylation ER Chaperone Pathways for alphα7 Nicotinic Receptor Assembly. Cell Rep, 2020. 32(6): p. 108025.
57. Guo, J., et al., PDCD4 Knockdown Induces Senescence in Hepatoma Cells by Up-Regulating the p21 Expression. Front Oncol, 2018. 8: p. 661.
58. Curtis, A., et al., Temporal variations of skin pigmentation in C57BL6 mice affect optical bioluminescence quantitation. Mol Imaging Biol, 2011. 13(6): p. 1114-23.

We claim:

1. A peptide comprising a first portion, a second portion and a third portion, wherein the first portion is a nicotinic acetylcholine receptor (nAChR) subtype selective portion of an α-neurotoxin, wherein the second portion is a portion of a rabies viral glycoprotein (RVG) peptide, and wherein the third portion is a nicotinic acetylcholine receptor (nAChR) subtype selective portion of an α-neurotoxin.

2. The peptide of claim 1, wherein the portion of the rabies viral glycoprotein (RVG) peptide is a cell penetrating peptide.

3. The peptide of claim 1, wherein the first portion comprises the amino acid sequence YRKMW (SEQ ID NO:7), YTKTW (SEQ ID NO:8) or a sequence with 60% identity to SEQ ID NO:7 or SEQ ID NO:8, the second portion comprises the amino acid sequence MPENPRLGTS (SEQ ID NO:2) or a sequence with 60% identity to SEQ ID NO:2, and the third portion comprises the sequence CDAFCSSRGKVVELG (SEQ ID NO:9), CDAFCSIRGKRVDLG (SEQ ID NO: 10) or a sequence with 60% identity to SEQ ID NO:9 or SEQ ID NO:10.

4. The peptide of claim 1, wherein the peptide comprises or consists of
i) the amino acid sequence YRKMWMPENPRLGTSCDAFCSSRGKVVELG (SEQ ID NO:11) or a sequence having at least 60% identity to SEQ ID NO:11, or
ii) the amino acid sequence having one or more amino acid substitutions at positions 3, 5, 14, 17, 19, 22, 23, or 26 of SEQ ID NO:11.

5. The peptide of claim 1, wherein the α-neurotoxin is α-bungarotoxin or α-cobratoxin.

6. The peptide of claim 1, wherein the portion of the RVG peptide is a portion of a challenge virus standard (CVS), Pasteur Vaccine (PV), or Sad-B19 strain.

7. The peptide of claim 1, wherein the an amino acid sequence having at least 60% identity to YRKMW (SEQ ID NO:7) comprises an amino acid substitution at position 3, 5, or both of YRKMW (SEQ ID NO:7).

8. The peptide of claim 1, wherein the amino acid sequence having at least 60% identity to CDAFCSSRGKVVELG (SEQ ID NO:9) comprises an amino acid substitution at position 4, 7, 8, 11, or a combination thereof of CDAFCSSRGKVVELG (SEQ ID NO:9).

9. The peptide of claim 1, wherein the nAChR subtype selective portion of an α-neurotoxin is specific for α7 and/or α9α10 nAChRs.

10. The peptide of claim 1, further comprising cargo including a therapeutic or molecular tag.

11. The peptide of claim 10, wherein the therapeutic or molecular tag are on the C- or N-terminal ends of the peptide.

12. The peptide of claim 10, wherein the therapeutic is siRNA, a viral vector, a nanoparticle, a liposome, a protein, or a compound.

13. The peptide of claim 12, wherein the viral vector is an adeno-associated viral (AAV) vector.

14. The peptide of claim 1, further comprising a non-naturally occurring amino acid.

15. The peptide of claim 14, wherein the non-naturally occurring amino acid is a non-naturally occurring arginine.

16. The peptide of claim 1, wherein the peptide is capable of greater than 80% inhibition of ACh-mediated currents at 100 μM peptide on no more than two ACh subtypes and less than 25% inhibition on the other nAChRs, and/or wherein the peptide is capable of greater than 80% inhibition of α7 and α9α10 nAChRs and less than 25% inhibition of β3α6β2α4β2, α6/α3β2β3, α4β2α5, α3β2, α3β2α3, α4β2, α3β4, α1β1δγ, and α1β1δε, and/or wherein the peptide has an IC₅₀ value in the nM-μM range for α7 and α9α10 receptors while maintaining less than 25% inhibition on the other subtypes.

17. A method of inhibiting α7 and/or α9α10 nAChR in a subject comprising administering one or more of the peptides of claim 1 to a subject in need thereof.

18. The method of claim 17, wherein the subject in need thereof has central nervous system (CNS) hypercholinergic tone.

19. The method of claim 18, wherein the subject having CNS hypercholinergic tone has chronic pain or depression.

20. A method of treating one of inflammation, chronic pain, and depression in a subject comprising administering a composition comprising the peptide of claim 1 to a subject in need thereof.


SEQUENCE LISTING (24 sequences)

SEQ ID NO: 1 - protein, 29 aa, synthetic construct
YTIWMPENPRLGTSCDIFTNSRGKRASKG

SEQ ID NO: 2 - protein, 10 aa, synthetic construct
MPENPRLGTS

SEQ ID NO: 3 - protein, 29 aa, synthetic construct
YTIWMPENPRPGTPCDIFTNSRGKRASNG

SEQ ID NO: 4 - protein, 29 aa, synthetic construct
YTIWMPENPRLGMSCDIFTNSRGKRASKG

SEQ ID NO: 5 - protein, 20 aa, synthetic construct
YRKMWCDAFCSSRGKVVELG

SEQ ID NO: 6 - protein, 20 aa, synthetic construct
YTKTWCDAFCSIRGKRVDLG

SEQ ID NO: 7 - protein, 5 aa, synthetic construct
YRKMW

SEQ ID NO: 8 - protein, 5 aa, synthetic construct
YTKTW

SEQ ID NO: 9 - protein, 15 aa, synthetic construct
CDAFCSSRGKVVELG

SEQ ID NO: 10 - protein, 15 aa, synthetic construct
CDAFCSIRGKRVDLG

SEQ ID NO: 11 - protein, 30 aa, synthetic construct
YRKMWMPENPRLGTSCDAFCSSRGKVVELG

SEQ ID NO: 12 - protein, 30 aa, synthetic construct
YTKTWMPENPRLGTSCDAFCSIRGKRVDLG

SEQ ID NO: 13 - protein, 20 aa, synthetic construct
YTKTWCDGFCSSRGKRIDLG

SEQ ID NO: 14 - protein, 20 aa, synthetic construct
FLKAQCDKFCSIRGPVIEQG

SEQ ID NO: 15 - protein, 20 aa, synthetic construct
FLKAQCDKFCPIRGPVIEQG

SEQ ID NO: 16 - protein, 20 aa, synthetic construct
FRKAQCDNFCHSRGPVIEQG

SEQ ID NO: 17 - protein, 20 aa, synthetic construct
FTKAFCDRWCSSRGPVIEQG

SEQ ID NO: 18 - protein, 17 aa, synthetic construct
MTTRTYYTPTRMKVSKS

SEQ ID NO: 19 - protein, 16 aa, synthetic construct
QKEVMEQSAGIMYRKS

SEQ ID NO: 20 - protein, 22 aa, synthetic construct
MTTLVTVEAEYPFNQSPVVTRS

SEQ ID NO: 21 - protein, 20 aa, synthetic construct
VTTATRVLSNTEDLPLVTKM

SEQ ID NO: 22 - protein, 4 aa, synthetic construct
YTIW

SEQ ID NO: 23 - protein, 15 aa, synthetic construct
CDIFTNSRGKRASKG

SEQ ID NO: 24 - DNA, 93 bp, synthetic construct
  source 1..93: mol_type = other DNA
tacagaaagatgtggatgcccgagaaccccagactgggcaccagctgcgacgccttctgcagcagcagaggcaaggtggtggagctgggctga